(19) United States
(12) Patent Application Publication
(10) Pub. No.: US 2006/0172958 A1
(43) Pub. Date: Aug. 3, 2006

(54) Phospholipid scramblase 3

(76) Inventors: Ruey-min Lee, Salt Lake City, UT (US); Qiang Dai, Salt Lake City, UT (US); Jun Chen, Salt Lake City, UT (US); Jihua Liu, Salt Lake City, UT (US)

Correspondence Address: Madson & Metcalf, Suite 900, 15 West South Temple, Salt Lake City, UT, 84101, US

(21) Appl. No.: 10/548,329

(22) PCT Filed: Mar. 15, 2004
(86) PCT No.: PCT/US04/07920

§ 371 Date: Sep. 7, 2005

(30) Foreign Application Priority Data
Mar. 13, 2003 (US) 60454494

(51) Int. Cl. A61K 48/00; C12Q 1/68; C07H 21/02

(52) U.S. Cl. 514/44; 435/6; 536/23.1

(57) ABSTRACT

Phospholipid scramblase 3 (PLS3) is a newly recognized member of a family of proteins responsible for phospholipid translocation between two lipid compartments. A novel isoform of PLS3 is identified and characterized herein. The function of PLS3 in mitochondria was disrupted, yielding an inactive mutant PLS3(F258V). Cells transfected with PLS3(F258V) exhibited reduced proliferative capacity that was unaffected by the presence of Na₃N. PLS3(F258V)-expressing cells exhibit abnormal mitochondrial metabolism and structure and were associated with decreased sensitivity to UV- and tBid-induced apoptosis, and diminished translocation of cardiolipin to the outer mitochondrial membrane. Cells transfected with wild-type PLS3 displayed increased sensitivity to apoptosis and enhanced cardiolipin translocation. These studies identify PLS3 as a regulator of mitochondrial structure and respiration, and cardiolipin transport in apoptosis.

### BACKGROUND OF THE INVENTION

Regulation of apoptosis, or programmed cell death, is critical for development and tissue homeostasis. Dysregulation of apoptosis is a key factor in neoplastic transformation. Reed, Dysregulation of apoptosis in cancer, J. Clin. Oncol., 17: 2941-2953, (1999); Ionov et al., Mutational inactivation of the proapoptopic gene BAX confers selective advantage during tumor clonal evolution, Proc. Natl. Acad. Sci. USA., 97: 10872-10877, (2000). Apoptotic cell death is characterized by a proteolytic caspase cascade that emanates from either an ‘extrinsic’ pathway, initiated by membrane-bound death receptors leading to activation of caspase-8, or an ‘intrinsic’ pathway triggered by DNA-damaging drugs and UV radiation leading to mitochondrial depolarization and subsequent activation of caspase-9. Green and Evan, A matter of life and death, Cancer Cell, 1: 19-30, (2002). Caspase activation leads to distinct morphological changes, including mitochondrial disintegration, followed by nuclear fragmentation, chromatin condensation, and cytoplasmic membrane blebbing. Cory and Adams, Matters of life and death: programmed cell death at Cold Spring Harbor, Biochim. Biophys. Acta, 1377: R25-R44, (1998); Kroemer and Reed, Mitochondrial control of cell death, Nat. Med., 6: 513-519, (2000).

An additional early morphological event in cells undergoing apoptosis is translocation of phosphatidylserine (PS) from the inner to the outer leaflet of the plasma membrane. Fadok et al., Exposure of phosphatidylserine oil the surface of apoptotic lymphocytes triggers specific recognition and removal by macrophages, J. Immunol, 148: 2207-2216, (1992); Bratton et al., Appearance of phosphatidylserine on apoptotic cells requires calcium-mediated nonspecific flip-flop and is enhanced by loss of the aminophospholipid translocase, J. Biol. Chem., 272: 26159-26165 (1997); and Martin et al., Early redistribution of plasma membrane phosphatidylserine is a general feature of apoptosis regardless of the initiating stimulus: inhibition by overexpression of Bcl-2 and Abl, J. Exp. Med., 182: 1545-1556, (1995). Externalized PS is recognized by macrophages, which rapidly remove apoptotic cells by phagocytosis. Fadok et al., Loss of phospholipids asymmetry and surface exposure of phosphatidylserine is required for phagocytosis of apoptotic cells by macrophages and fibroblasts, J. Biol. Chem., 276: 10781-1077 (2001); Fadok et al., A receptor for phosphatidylserine-specific clearance of apoptotic cells, Nature,405: 85-90 (2000). Mitochondria are central integrators of most apoptotic pathways. Brenner and Kroemer, Mitochondria—the death signal integrators, Science, 289: 1150-1151, (2000). Activation of the intrinsic pathway causes mitochondrial release of a number of pro-apoptotic molecules including cytochrome c, endonuclease G (Parrish et al., Mitochondrial endonuclease G is important for apoptosis in C. elegans, Nature, 412: 90-94 (2001); Li et al., Endonuclease G is an apoptotic DNase when released from mitochondria, Nature, 412: 95-99, (2001), SMAC/Diablo (Du et al., Smac, a mitochondrial protein that promotes cytochrome c-dependent caspase activation by eliminating IAP inhibition [In Process Citation], Cell, 102: 33-42, (2000); Verhagen et al., Identification of DIABLO, a mammalian protein that promotes apoptosis by binding to and antagonizing IAP proteins [In Process Citation], Cell, 102: 43-53 (2000)), and apoptosis-inducing factor (Susin et al., Molecular characterization of mitochondrial apoptosis-inducing factor, Nature, 397: 441-446, (1999)).

In addition, multiple pro-apoptotic regulators including Bax, Bad, Bid, Bim, p53, JNK, PKC-y, nuclear receptor TR3 (Brenner and Kroemer, Apoptosis. Mitochondria—the death signal integrators, Science, 289: 1150-1151 (2000); Li et al., Cytochrome c release and apoptosis induced by mitochondrial targeting of nuclear orphan receptor TR3, Science, 289: 1159-1164 (2000)), and the Peutz-Jegher gene product LKB1 (Karuman et al., The Peutz-Jegher gene product LKB1 is a mediator of p53-dependent cell death, Mol. Cell, 7: 1307-1319, (2001)) are translocated to mitochondria during apoptosis. In some cell types, the extrinsic pathway is linked to the intrinsic pathway via activation of caspase-8, which causes NH₂-terminal cleavage of Bid to generate tBid (Luo et al., Bid, a Bcl2 interacting protein, mediates cytochrome c release from mitochondria in response to activation of cell surface death receptors, Cell, 94: 481-490, (1998)). The active tBid fragment is N-myristoylated (Zha et al., Posttranslational N-myristoylation of BID as a molecular switch for targeting mitochondria and apoptosis, Science, 290: 1761-1765, (2000)) and then localizes to mitochondria through a positive interaction with cardiolipin (CL; Lutter et al., Cardiolipinprovides specificity for targeting of tBid to mitochondria, Nat. Cell Biol., 2: 754-761, (2000)). Activated tBid induces CL-dependent activation of Bax and Bak to form cytochrome c channels during apoptosis. Kuwana et al., Bid, Bax, and lipids cooperate to form supramolecular openings in the outer mitochondrial membrane, Cell, 111: 331-342, (2002). In the absence of Bax and Bak, tBid is unable to induce cytochrome c release. Wei et al., tBID, a membrane-targeted death ligand, oligomerizes BAK to release cytochrome c, Genes Dev., 14: 2060-2071, (2000); Wei et al., Proaptoptic BAX and BAK: a requisite gateway to mitochondrial dysfunction and death, Science, 292: 727-730, (2001); and Korsmeyer, Pro-apoptotic cascade activates BID, which oligomerizes BAK or BAX into pores that result in the release of cytochrome c, Cell Death Differ., 7: 1166-1173, (2000).

Phospholipid scramblases (PLS) are enzymes responsible for bidirectional movement of phospholipids (Bevers et al., Lipid translocation across the plasma membrane of mammalian cells, Biochim. Biophys. Acta, 1439: 317-330, (1999)), and four PLS family members have been identified (Wiedmer et al., Identification of three new members of the phospholipids scramblase gene family, Biochim. Biophys. Acta, 1467: 244-253, (2000)). PLS1 is located in the plasma membrane and is responsible for translocation of phospholipids between the inner and outer leaflets. Zhou et al., Molecular cloning of human plasma membrane phospholipids scramblase, A protein mediating transbilayer movement of plasma membrane phospholipids, J. Biol. Chem., 272: 18240-18244 (1997). Although apoptotic PS translocation was unaltered in PLS1-deficient mice (Zhou et al., Normal hemostasis but defective hematopoietic response to growth factors in mice deficient in phospholipid scramblase 1, Blood, 99: 4030-4038, (2002)), the role of PLS1 in apoptosis remains unclear given the presence of additional enzymes associated with plasma membrane phospholipid translocation such as aminophospholipid translocase. (Bevers et al., Lipid translocation across the plasma membrane of mammalian cells, Biochim Biophys. Acta, 1439: 317-330, (1999); Zhao et al., Level of expression of phospholipids scramblase regulates induced movement of phosphatidylserine to the cell surface, J. Biol. Chem., 273: 5503-5505, (1998); and Williamson et al., Phospholipid scramblase activation pathways in lymphocytes, Biochemistry, 40: 8065-8072 (2001).

PLS family members contain a conserved calcium-binding motif, and Zhou et al. (Identity of a conserved motif in phospholipids scramblase that is required for Ca2+-accelerated transbilayer movement of membrane phospholipids, Biochemistry, 37: 2356-2360, (1998)) found that mutation of residues in this region of PLS1 completely eliminated enzymatic activity. PLS1 is phosphorylated at Thr-161 by PKC-8, which translocates to the plasma membrane during apoptosis; in addition, PLS1 is phosphorylated at Tyr-69/74 by c-Abl kinase. Sun et al., c-Abl tyrosine kinase binds and phosphorylates phospholipids scramblase 1, J. Biol. Chem., 276: 29894-28990, (2001). The role of calcium binding and these various phosphorylation events in PLS1 activation and regulation, however, remain to be determined.

A newly identified member of the scramblase family, designated PLS3, is localized to the mitochondria rather than plasma membrane. Liu et al., Phospholipid scramblase 3 is the mitochondrial target of protein kinase C δ-induced apoptosis, Cancer Res., 63: 1153-1156, (2003). However, little is known regarding the physiological function of PLS3 in mitochondria. It has been shown previously that PLS3, like PLS1, is phosphorylated by PKC-δ. Id. A mitochondrial targeted PKC-δ dramatically enhanced susceptibility to apoptosis in cells overexpressing PLS3, suggesting that PLS3 is the direct mitochondrial effector of PKC-δ-induced apoptosis. Id.

Members of the PKC family of kinases play diverse roles in cell signaling, proliferation, apoptosis, and other cellular processes. Parker and Dekker, Protein Kinase C., Georgetown: Landes Bioscience, 1997; and Ohno, The distinct biological potential of PKC isotypes, Protein Kinase C., pp. 75-95, Georgetown: Landes Bioscience, 1997. PKC-δ is particularly associated with apoptosis. Denning et al., Caspase activation and disruption of mitochondrial membrane potential during UV radiation-induced apoptosis of human keratinocytes requires activation of protein kinase C., Cell. Death Differ., 9: 40-52, 2002; Fujii et al., Involvement of protein kinase Cδ (PKCδ) in phorbol ester-induced apoptosis in LNCαP prostate cancer cells, Lack of proteolytic cleavage of PCKδ, J. Biol. Chem., 275: 7574-7582 (2000); Li et al., Protein kinase Cδ targets mitochondria, alters mitochondrial membrane potential, and induces apoptosis in normal and neoplastic keratinocytes when overexpressed by an adenoviral vector, Mol. Cell. Biol., 18: 8574-8558 (1999); Majumder et al., Mitochondrial translocation of protein kinase Cδ in phorbol ester-induced cytochrome c release and apoptosis, J. Biol. Chem., 275: 21793-21796 (2000); Mandil et al., Protein kinase Cα and protein kinase Cδ play opposite roles in the proliferation and apoptosis of glioma cells, Cancer Res., 61: 4612-4619 (2001); and Yoshida and Kufe, Negative regulation of the SHPTP1 protein tyrosine phosphatase by protein kinase Cδ in response to DNA damage, Mol. Pharmacol., 60: 1431-1438 (2001). PKC-δ is present in the cytoplasm, and translocates to various organelles, including the nucleus, mitochondria, and the plasma membrane, on apoptotic stimulation. Id. One substrate of PKCδ is PLS1 in the plasma membrane. Frasch et al., Regulation of phospholipids scramblase activity during apoptosis and cell activation by protein kinase Cδ, J. Biol. Chem., 275: 23065-23073 (2000). However, the consequence of PLS1 phosphorylation and how PLS1 contributes to apoptosis remain elusive. PKC-δ is activated during apoptosis by caspase-mediated cleavage to become catalytically active. Ghayur, et al., Proteolytic activation of protein kinase C δ by an ICE/CED 3-like protease induces characteristics of apoptosis, J. Exp. Med., 184:2399-2404 (1996). This catalytic active fragment of PKC-δ translocates to the mitochondria to induce apoptotic events. Inhibition of PKC-δ blocks the disruption of the mitochondrial transmembrane potential. These results indicate that although PKC-δ plays an important role in triggering apoptosis through a mitochondrion-dependent pathway. However, the substrate of PKC-δ—in the mitochondria remains unidentified.

The function of PLS is to associate translocated phospholipids bidirectionally between two compartments. Bevers et al., Transmembrane phospholipids distribution in blood cells: control mechanisms and pathophysiological significance, Biol. Chem., 379: 973-986 (1998) and Sims and Wiedmer, Unraveling the mysteries of phospholipids scrambling, Thromb. Haemost., 85: 266-275 (2001). PLS1 translocates phospholipids between the inner and outer leaflets of the plasma membrane, which is asymmetric in lipid composition. Although PS is translocated to the outer leaflet during apoptosis, it is still unclear whether PLS1 is responsible for this translocation. Fadok et al., Exposure of phosphatidylserine on the surface of apoptotic lymphocytes triggers specific recognition and removal by macrophages, J. Immunol., 148: 2207-2215 (1992); Fadeel et al., Phosphatidylserine exposure during apoptosis is a cell-type-specific event and does not correlate with plasma membrane phospholipids scramblase expression, Biochem. Biophys. Res. Commun., 266: 504-511 (1999); Bratton et al., Appearance of phosphatidylserine on apoptotic cells requires calcium-mediated nonspecific flip-flop and is enhanced by loss of the aminophospholipid translocase, J. Biol. Chem., 272: 26159-26165 (1997); Martin et al., Early redistribution of plasma membrane phosphatidylserine is a general feature of apoptosis regardless of the initiating stimulus: inhibition by overexpression of Bcl-2 and Abl., J. Exp. Med., 182: 1545-1556 (1995); and Zhou et al., Molecular cloning of human plasma membrane phospholipids scramblase. A protein mediating transbilayer movement of plasma membrane phospholipids, J. Biol. Chem., 272: 18240-18244 (1997).

Bcl-B is the human orthologue of mouse BOO/Diva, a member of Bcl-2 family similar to avian NR13. Avian NR13 was originally identified as a v-src-activated gene (Gillet et al. 1995), and overexpression of NR13 protein protected Baf-3 cells from IL-3 withdrawal-induced apoptosis (Mangeney et al. 1996). NR13 plays a major role in regulation of chicken bursal apoptosis. NR13 protected a bursa-derived cell line, DT40, from serum-deprivation-induced apoptosis, and the level of NR13 correlated with bursal cell survival in vivo. Using a bursal transplantation model, it was demonstrated that one physiological function of NR13 is to protect the bursal stem cells from programmed elimination (Lee et al. 1999). Searching for mammalian homologues of avian NR13, two groups identified mouse BOO/Diva through EST database analysis (Inohara et al. 1998; Song et al. 1999). It has a unique tissue distribution, expressed in all embryonic tissues but only in adult ovary. Protection from or promotion of cell death by BOO/Diva was cell-type dependent. BOO/Diva interacted with Apaf-1; however, the physiological significance of this interaction is unclear (Inohara et al. 1998; Song et al. 1999). Bcl-B is highly conserved between mouse and human, and its anti- or pro-apoptotic effect is significantly weaker compared with Bcl-2 or Bcl-xL. Bcl-B interacts with Bcl-2, Bcl-xL and Bax, but not Bak (Ke et al. 2001). Bcl-B is not completely localized in mitochondria, but translocates to mitochondria upon induction of cell death by microtubule-interfering agents. Bcl-B gene was mapped in chromosome 15q21, and deletion was identified in some primary cervical cancers (Lee et al. 2001).

The biochemical functions of Bcl-2 family in mitochondria are far from clear. The crystal structure of Bcl-xL (Muchmore et al. 1996) suggested that Bcl-2 family members could form channels, and regulate the mitochondrial transmembrane potential (Δψ) and the release of cytochrome c upon pro-apoptotic stimulation (Shimizu et al. 1999; Vander Heiden and Thompson 1999; Shimizu et al. 2000; Shimizu and Tsujimoto 2000). The regulation of mitochondrial transmembrane potential (Δψ) is mediated through a transmembrane permeability pore complex (Shimizu et al. 1999). This multiprotein complex contains many components, including voltage-dependent anion channel (VDAC) and peripheral benzodiazepine receptor (PBR) on the outer membrane of mitochondria, adenine nucleotide translocator (ANT) on the inner membrane, and a matrix protein cyclophilin D (CphD). Shimizu et al. used liposomes carrying VDAC to show that Bax and Bak accelerated the opening of VDAC, while Bcl-xL closed VDAC through direct interaction with VDAC.

Using a BH4 domain peptide of Bcl-xL, they effectively prevented apoptotic cell death induced by etoposide treatment, indicating that BH4 domain of Bcl-xL was critical for this function (Shimizu et al. 2000). On the other hand, the BH3-only Bcl-2 family members induce release of cytochrome c without Δψ loss, and do not directly modulate VDAC activity (Shimizu and Tsujimoto 2000). These results demonstrated two different functions of the mitochondrial transmembrane pore complex, i.e. maintaining mitochondrial transmembrane potential (Δψ) and serving as a transmembrane protein channel, and they are regulated through different domains of Bcl-2 family members.

Cardiolipin has recently drawn attention as a mitochondrial phospholipid involved in apoptosis. This dimeric phospholipid contains two phosphatidyl molecules linked by a glycerol moiety, thus in total four fatty acyl chains and two phosphate groups (Hoch 1992). Cardiolipin is mainly located at the inner membranes of mitochondria (Krebs et al. 1979) and is required for oxidative phosphorylation and high energy electron transport (Jiang et al. 2000; Schlame et al. 2000). Cardiolipin interacts with cytochrome c, and peroxidation of cardiolipin dissociates cytochrome c from cardiolipin (Shidoji et al. 1999; Nomura et al. 2000).

Studies of cardiolipin were carried out with a cardiolipin-specific fluorescence dye NAO, nonyl-acridine orange (Garcia Fernandez et al. 2000). NAO has a fluorescence emission at 625 nm with 2:1 stoichiometric interaction to cardiolipin, and at 525 nm with 1:1 interaction. Measuring the amount of cardiolipin can be done with NAO by flow cytometry analysis (Garcia Fernandez et al. 2000). When cells become apoptotic, NAO binding decreases secondary to peroxidation of cardiolipin, a process that contributes to the release of cytochrome c (Shidoji et al. 1999). Another function of cardiolipin was recently identified as the mitochondrial target for tBid, which is cleaved from Bid upon apoptotic stimulation and induces activation of caspase 9 along with cytochrome c and Apaf-1 (Lutter et al. 2000).

Apoptosis is a center of much attention in current efforts to establish more effective tailored treatments of cancer, as well as many other conditions, including neurodegenerative disorders, and disorders affecting the immune system. Research efforts often center on locating cellular targets for the regulation of apoptosis. Some benefit may be obtained from the discovery of targets and methods useful for inducing apoptosis or preventing it. In cancer, it is desirable to discover or provide compounds and/or methods for regulating apoptosis in targeted cancer cells as a treatment method. Such compounds and methods have proven elusive, however. Similarly, in neurodegenerative disorders and disorders affecting the immune system, it would be beneficial to provide compounds and/or methods for arresting apoptotic processes that proceed in an uncontrolled fashion, causing damage to nervous tissue.

Such gene targets, and methods for inducing and preventing apoptosis are provided herein.

### BRIEF SUMMARY OF THE INVENTION

The present invention relates in part to a gene which is involved in processes associated with apoptosis. More specifically, the present invention relates to the phospholipid scramblase 3 gene (hereinafter “PLS3”) involved in the modification of mitochondrial membranes during apoptosis.

Phospholipid scramblase 3 (PLS3) is a newly recognized member of a family of proteins responsible for phospholipid translocation between two lipid compartments. An alternatively-spliced form of PLS3 (herein referred to as “PLS3α”, SEQ ID NO: 2) was identified using yeast two-hybrid screening to search for binding partners of Boo/Diva, a Bcl-2 family member. This PLS3 isoform encodes a translated product (SEQ ID NO: 1) that differs at the last 17 amino acids from the PLS3 isoform known and found in GenBank (herein referred to as “PLS3β”, SEQ ID NO: 4). The sequences of the translated products, SEQ ID NO: 1, SEQ ID NO: 3 are compared with those of a mouse, drosophila, and C. elegans in FIG. 1. The longer message (PLS3β) is expressed in heart and skeletal muscle; while the short transcript (PLS3α) is expressed in spleen, liver, kidney and placenta. See, e.g., FIG. 2.

In contrast to PLS1 that is present in the plasma membrane, both PLS3α and PLS313 are localized in mitochondria. Computer prediction suggests that they have two transmembrane domains rather than the single transmembrane domain found in PLS1. Proteinase K digestion and epitope mapping were used to study the topology of native PLS3α of mouse liver mitochondria. Without being limited to any one theory, these techniques appeared to yield the conclusion that PLS3α crosses both mitochondrial inner and outer membranes. This topology suggests that PLS3 may be responsible for transferring phospholipids between the two mitochondrial membranes.

The conserved calcium-binding motif of PLS3 was disrupted in the studies discussed herein to evaluate the function of PLS3 in mitochondria. This process yielded an inactive mutant PLS3(F258V). Cells transfected with PLS3(F258V) exhibited reduced proliferative capacity that was unaffected by the presence of Na₃N. Mitochondrial analysis revealed that PLS3(F258V)-expressing cells have decreased mitochondrial mass shown by lower cytochrome c and cardiolipin content, poor mitochondrial respiration, and reduced oxygen consumption and intracellular ATP. In contrast, wild-type PLS3-transfected cells exhibit increased mitochondrial mass and enhanced respiration. Electron microscopic examination revealed that the mitochondria in PLS3(F258V)-expressing cells have densely packed cristae, and are fewer in number and larger than those in cells transfected with wild-type PLS3.

It was also found that the abnormal mitochondrial metabolism and structure in PLS3(F258V)-expressing cells was associated with decreased sensitivity to UV- and tBid-induced apoptosis, and diminished translocation of cardiolipin to the outer mitochondrial membrane. By contrast, wild-type PLS3-transfected cells displayed increased sensitivity to apoptosis and enhanced cardiolipin translocation. These studies appear to identify PLS3 as a regulator of mitochondrial structure and respiration, and cardiolipin transport in apoptosis.

In addition to the above, it was noted that the deletion mutant of PLS3 disrupted mitochondrial transmembrane potential and induced cell death. Incubation of purified PLS3 protein with mitochondria in vitro resulted in disruption of mitochondrial transmembrane potential and cytochrome c release. In phospholipid transfer assays, PLS3 was noted to have a substrate specificity to cardiolipin. UV irradiation and overexpression of PLS3 in 293 cells increase the percentage of cardiolipin at the outer membrane of mitochondria. Co-expression of Bcl-B and PLS3 enhanced the pro-apoptotic effect of Bcl-B. This pro-apoptotic effect of Bcl-B, however, is inhibited by an inactive mutant of PLS3. These results indicate that PLS3 is a downstream effector of Bcl-B.

These results also make PLS3 a target for research and discovery of compounds for the induction of apoptosis or for blocking apoptosis. Indeed, the discovery and isolation of such a gene allows the screening of compounds to isolate molecules capable of up- or down-regulating the production of PLS3 in a cell, and thus inducing or blocking apoptosis.

PKC-δ translocates to mitochondria during apoptosis, but its mitochondrial target remains unclear. It was found that PKC-δ physically interacted with and phosphorylated phospholipid scramblase 3 (PLS3) after UV irradiation. PLS3 is a high affinity substrate for PKC-δ in vitro with the km at 10.5 nM. Cells expressing wild-type PLS3 became apoptotic upon phorbol ester stimulation; while the control cells did not. Expression of a mitochondrial targeted PKC-δ enhanced apoptosis more prominently in HeLa-PLS3 cells than control HeLa cells and HeLa cells expressing an inactive PLS3 mutant. These results indicate that PLS3 is a downstream effector of PKC-δ in the mitochondria.

Thus, the invention provides a novel target for the regulation of cellular apoptosis. In part, the invention provides a novel isoform of PLS3, namely PLS3α (SEQ ID NO: 2, product SEQ ID NO: 1). The invention further provides methods of rendering a cell resistant to apoptosis. In addition, the invention provides methods of sensitizing a cell to apoptosis. Each of these is discussed in greater detail below.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1: Clustal alignment of PLS3 proteins of human (PLS3α, PLS3β), mouse, Drosophila and C. elegans. The amino acids that are identical among four species are marked by “*”. The amino acids that are similar are marked by “:”. The differences between PLS3α and PLS3β are highlighted in bold.

FIG. 2: (a) Physical interaction of PLS3α with Bcl-B. 293T cell lysates from PLS3α and EGFP-Bcl-B overexpressing cells were immunoprecipitated with polyclonal PLS3 antibody and subjected to Western analysis with EGFP monoclonal antibody. IP control was performed with pre-immune serum. (b) Tissue distribution of PLS3. Northern analysis of PLS3 was performed in human 12-lane MTN blots with PLS3 cDNA as probe. The probe with 2×10⁶ cpm/ml activity was used for hybridization. PLS3 blots were exposed for 2 days, and actin control was exposed for 12 hours. Two arrows indicate the two alternatively spliced forms of PLS3. The lower is PLS3α and the upper is PLS3β.

FIG. 3. Subcellular localization of PLS3 with EGFP-PLS3 fusion protein. (a) EGFP-PLS3α was transfected into 293 cells, and MitoTracker dye was then added for localization. Cells were visualized under confocal microscope, revealing that EGFP-PLS3α corresponds with MitoTracker. EGFP-PLS3β exhibits a similar pattern (not shown). (b) EGFP-ΔPLS3 was transfected into 293 cells for colocalization with MitoTracker similar to (a). Three cells that express EGFP failed to pick up the red MitoTracker dye. (c) Subcellular fractionation for Western analysis with PLS3 polyclonal antibody. Cells with PLS3α overexpression were harvested and subjected to differential centrifugation to obtain nuclei, mitochondria, microsomes and cytosols. Equal amounts of protein (20 μg) were loaded into gel for separation, and Western analysis was done with PLS3 antibody. Mitochondrial proteins from mouse liver and 293 cells were also analyzed with the same antibody, with purified PLS3 used as a positive control.

FIG. 4. Topology of PLS3 in mitochondria. (a) Six potential topologies of PLS3 protein in mitochondria are illustrated. The arrow direction is from N to C terminus of the protein. The solid bars represent antibody recognition sites. TM: transmembrane domain. (b) Western analysis of mitochondria after protease K digestion with PLS3 Ab-1 (left) or Ab-N (right). The sizes of PLS3 or its cleavage products are indicated by MW markers. (c) Mitoplasts were digested with protease K as in (b), followed by Western analysis with Ab-1. OM lane is the Western analysis of outer membrane fraction. (d) Mitoplasts were digested with protease K followed by Western analysis with Ab-N. (e) Fluorescence intensities of epitope mapping by Ab-1 and Ab-N. Purified mitochondria or mitoplasts were incubated with Ab-1 or Ab-N, followed by secondary antibody conjugated with FITC. The fluorescence intensities of the washed pellets were read by a microplate reader.

FIG. 5. Disruption of mitochondrial transmembrane potential and release of cytochrome c by PLS3β in vitro. (a) Purified PLS313 protein (0.1, 0.5 or 1.0 μg) was incubated with freshly isolated mouse mitochondria (100 μg by protein) and analyzed for Δψ with Rhodamine 123. Calcium (100 or 200 μM) was used as positive control to disrupt Δψ. The fluorescence intensity at 590±35 nm was measured by microplate reader. NC is negative control of mitochondria treated with buffer only. (b) Supernatants of the mixtures of PLS3 and mitochondria were analyzed by Western with monoclonal antibody against cytochrome c. The first two lanes are supernatant and mitochondrial pellet treated with buffer only for control.

FIG. 6. In vitro phospholipid transfer assays. (a) Phospholipid transfer assays with PC, PE and cholesterol. Liposomes containing ¹⁴C-labeled phospholipids were incubated with mouse liver mitochondria along with either BSA or PLS3 (20 μg) for 20 min. Mitochondria were washed and counted. Each bar is an average of three experiments. (b) Cardiolipin transfer assays were performed by incubating cardiolipin liposomes, mitochondria and either BSA or PLS3 protein. The washed mitochondria were then mixed with 30 μM NAO for measurement of fluorescence intensities at 590 nm. NC: negative control with buffer only. Each bar is an average of three experiments.

FIG. 7. Redistribution of cardiolipin at the outer membranes of mitochondria after UV irradiation and Bcl-B expression. (a) 293 cells with or without UV irradiation were fixed and stained with various concentrations of NAO. Fluorescent intensities of NAO were measured by a microplate reader at 530±25 nm and 680±30 nm. The curve in 680±30 nm was converted to percentage relative to the maximal fluorescence intensity at 35 μM NAO in longitudinal axis. The positions of the shoulders on each curve are marked by arrowheads. Each point is the average of four measurements. (b) 293-PLS3 cells were analyzed as in (a). (c) 293-PLS3 cells were transfected with pcDNA-Bcl-B and subjected to similar analysis as in (a) to determine the percentage of cardiolipin at the outer membrane of mitochondria. Arrowheads point to the shoulder of the curves.

FIG. 8. PLS3 is a downstream target of Bcl-B. (a) Synergism of Bcl-B and PLS3 in enhancing apoptosis. 293T cells were transfected with equal amount of control, Bcl-B, PLS3 or both PLS3 and Bcl-B expression plasmids, and pictures were taken 48 hours after transfection. The apoptotic cells appear as round and shining circles. (b) Model of Bcl-B and PLS3 interaction and activation of apoptosis in mitochondria.

FIG. 9: (a) Genomic structure of PLS3 and the two alternatively spliced forms. The open boxes are exons, and the solid boxes represent the coding region. (b) Northern analysis of PLS3 in multiple human tissue blot. The radioactive probes at 2×10⁶ cpm/ml were used for hybridization, and the PLS3 blot was exposed for 24 hours and the actin blot was exposed for 8 hours. The messages of PLS3α and PLS3β were indicated by arrows.

FIG. 10. Interaction of PLS3 with hBoo and Bcl-2. (a) HEK293 cells were transfected with pEGFP-hBoo or pEGFP control and IP was performed with PLS3 antibody. The Western blot was probed with EGFP antibody. (b) HeLa and HeLa-Bcl-2 cells were immunoprecipitated with PLS3 antibody, and the Western blot was probed with Bcl-2 antibody. The positions of the Bcl-2 protein are indicated with an arrow. (c) Subcellular fractionation for Western analysis with PLS3 antibody. Mouse liver were harvested and subjected to differential centrifugation to obtain nuclei, mitochondria, microsomes and cytoplasm. Equal amounts of protein (20 μg) were loaded into the gel for separation, and Western analysis was done with PLS3 antibody. Same blot was probed with VDAC, tubulin antibody for control. (d) PLS3 is integrated in the mitochondria. Isolated mouse liver mitochondria were washed with 0.1 M Na₂CO₃, and the supernatants and pellets were analyzed with PLS3 antibody in Western blot.

FIG. 11. PLS3 is present in mitochondria. Co-localization of EGFP-PLS3 with mitochondrial dye MitoTracker Red. HEK293 cells were transfected with EGFP-PLS3-α expression vector and stained with MitoTracker Red (100 μM). The EGFP-PLS3-β displayed an identical pattern. Control EGFP had a diffuse cytoplasmic pattern.

FIG. 12. Topology of PLS3 in the mitochondria. (a) Six potential topologies of PLS3 in mitochondria given that PLS3 has two TM domains. The amino acids of the TM domains were numbered. Two epitopes that were used to raise antibodies were marked by solid bars (Ab-N and Ab-1). (b) Purified mitochondria were subjected to limited proteinase K digestion followed by Western blot analysis with PLS3 Ab-1. (c) Mitochondria were subjected to limited proteinase K digestion at various temperatures or in a time course from 0-30 minutes. The digested products were analyzed by PLS3 Ab-1. The lower arrows indicate the digested products. The first lane of temperature study is undigested control. (d, e) Proteinase K digestion of mitoplasts. Mitoplasts were digested at various concentrations of proteinase K. Western analysis was performed with Ab-1 (d) and Ab-N (e). The positions of PLS3 and its degradation products are marked. The molecular weight standards are indicated in left. (f) Epitope mapping of PLS3 with Ab-1 and Ab-N. Mitochondria or mitoplasts were incubated with Ab-1 or Ab-N followed by a secondary antibody conjugated with FITC. PK 0 or 1.0: No proteinase K digestion or digested with 1.0 μg before incubation with PLS3 antibody. The fluorescence of the washed pellets was next measured. NC: negative control by incubating mitochondria with secondary antibody only.

FIG. 13. Stably-transfected cell lines expressing wild-type PLS3 or mutant PLS3(F258V). (a) G418-resistant clones of HEK293 cells transfected with pcDNA, pcDNA-PLS3 or pcDNA-PLS3(F258V) were harvested, and whole cell lysates analyzed by Western blotting with anti-PLS3 antibody. (b) 293-vector, 293-PLS3 and 293-PLS3(F258V) cells were fractionated, and mitochondrial (M) and cytoplasmic (C) fractions were analyzed by Western blotting with antibodies against PLS3, VDAC and tubulin. (c) Growth curves of the 293-vector, 293-PLS3 and 293-PLS3(F258V) cells under normal growth conditions. Cells were plated on day 0, and counted at days 1, 2 by trypan blue exclusion. (d) Growth curves in the presence of Na₃N at three different concentrations.

FIG. 14. Overexpression of mutant PLS3 reduces mitochondrial mass, potential, and cytochrome c and CL content. A. 293-vector, 293-PLS3, and 293-PLS3(F258V) cells were incubated with JC-1 dye as indicated and green (left panel) and red (right panel) fluorescence were determined by flow cytometry. B. Flow cytometry curves for 293-vector, 293-PLS3, and 293-PLS3(F258V) cells stained with Rhodamine 123. The left panel shows the decrease of mitochondrial potential by treatment of 293-vector cells with 20 AM antimycin A for 6 h. The right panel shows the mitochondrial potential determined by Rhodamine 123. C. Western blotting of whole cell lysates from 293-vector (lane 1), 293-PLS3 (lane 2), and 293-PLS3(F258V) cells (lane 3). The blot was re-probed with antibodies to VDAC and tubulin for controls. D. 293-vector, 293-PLS3, and 293-PLS3(F258V) cells were stained with 10-N-nonyl-3,6-bis(dimethylamino) acridine orange (NAO), and fluorescence intensities at 570 nm were measured. Error bars, SDs from five independent measurements. E. Quantitative PCR analysis of mitochondrial NADH dehydrogenase in 400 ng of whole cell genomic DNA. Curves 1-3, different concentrations of standards; other curves, quantification of 293-vector, 293-PLS3, and 293-PLS3(F258V) in triplicates. The crossing points (in cycle numbers) of the three cells are indicated.

FIG. 15. Analysis of mitochondrial respiration. (a) The ATP concentrations of the HEK293-vector, HEK293-PLS3, and HEK293-PLS3(F258V) cells were measured by luciferase assays. The results are the averages of five experiments. (b) The oxygen consumption of the cells was measured with oxygen electrode by dissolved oxygen meter as described in methods. Mitochondria (50 μg) were isolated from HEK293-vector, HEK293-PLS3 and HEK293-PLS3(F258V) cells and placed in the chamber of Mitocell. The state 4 respiration was induced with succinate at a final concentration of 7 mM, and oxygen concentrations in the chamber were monitored for 2 min. ADP was then added into the same chamber to a final concentration of 150 μM to measure oxygen consumption in state 3 respiration for another 6 min. (c) Oxygen consumption in states 3 and 4 respiration. The slopes of each curve in b were determined and the oxygen consumption rates (pmole/min/μg mitochondrial protein) were calculated. The results are the averages of 3 independent experiments.

FIG. 16. Electron microscopic examination of the HEK293-vector, HEK293-PLS3, and HEK293-PLS3(F258V) cells. All are 33,047×fold magnification.

FIG. 17. UV irradiation of cells overexpressing PLS3 or PLS3(F258V) mutant. (a) HeLa, HeLa-vector, HeLa-PLS3 and HeLa-PLS3(F258V) cells were irradiated with UV at 4 J/m²/sec for 2 min followed by staining with MTT as described in methods. Shown are the averages of 5 independent measurements. (b) Cells were irradiated with UV as in (a), and the percentages of apoptosis was analyzed by annexin V-PE binding as described in methods. The percentages of annexin V-PE positive cells are indicated. (c) Cells were treated with or without UV and analyzed by JC-1. JC-1 is shown on the left and JC-1 red is shown on the right.

FIG. 18. PLS3 regulates CL content and apoptotic translocation. A. Mitochondria were fractionated into IM and OM, and the percentages of enzyme activities of MAO and MDH in the mitochondrial OM and IM fractions were determined. B. 293-vector cells were labeled with [³²P]Pi, UV-treated, and then mitochondrial IM and OM were isolated and lipids extracted for TLC analysis. Migration of phosphatidylcholine (PC), phosphatidylethanolamine (PE), and CL was established by nonradioactive standards followed by iodine staining (standards). C. ³²P-labeled lipids were analyzed from equal amounts of mitochondria from 293-PLS3 or 293-PLS3(F258V) cells with or without UV irradiation as in B. D. Shown are percentages of CL present in the OM of unirradiated (open bars) and UV-treated (shaded bars) 293-vector, 293-PLS3, and 293-PLS3(F258V) cells, derived from the ratio of OM to the sum of OM and IM. Error bars, SDs from three experiments.

FIG. 19. Effects of PLS3 on tBid-induced cytochrome c and SMAC release. A. Mitochondria (60 μg) isolated from HeLa-vector (lanes 1 and 4), HeLa-PLS3 (lanes 2 and 5), and HeLa-PLS3(F258V) cells (lanes 3 and 6) were incubated with buffer (lanes 1-3) or recombinant tBid (0.6 μg; lanes 4-6) for 20 min at 37° C. The supernatants and pellets were subjected to Western analysis for cytochrome c, SMAC, and VDAC. B. The bands of cytochrome c and SMAC in A were quantified by densitometry and the percentages of tBid-induced mitochondrial release are shown.

FIG. 20. (a) PLS3 is phosphorylated at threonine. HEK293 or HEK293-PLS3 cells were treated with or without UV. Cell lysates were immunoprecipitated with PLS3 antibody. Western blot analysis was then performed with PS, PT, PY, PLS3 and PKC-δ antibodies. (b) UV irradiation induces apoptosis requires PKC-δ. HEK293 cells were treated with UV in the presence or absence of PKC-δ inhibitor, rottlerin (10 μM). Cells were harvested 4 hours later and fractionated to isolate mitochondria and cytosols. Fractions were analyzed with Western blot with cytochrome c, VDAC, tubulin antibodies. In the bottom two panels, the mitochondrial fractions were further washed with mitochondrial isolation buffer to remove rottlerin. The washed mitochondria were then incubated with 100 nM PMA to activate PKC-δ to determine if they were translocated to the mitochondria. After 20 minutes incubation, the mitochondria were separated from the supernatants. The supernatants and pellets were analyzed for cytochrome c.

FIG. 21. Enzyme kinetics of PKC-δ phosphorylating PLS3. a, in vitro phosphorylation of PLS3 with recombinant PKC-δ in a time course from 0 to 25 min. Recombinant PLS3 protein (1 μg) and [γ-32P]ATP were used as substrates, and recombinant PKC-δ was used as kinase for each in vitro phosphorylation reaction. b, kinetics study of PKC-δ toward PLS3 was performed using various concentrations of PLS3 and equal amount of PKC-δ for each reaction. The reaction was stopped after 20 min of incubation and analyzed by gel electrophoresis. The radioactivity of each PLS3 protein was determined by density analysis and plotted against the concentrations of the PLS3 protein. The plot in c is the double-reciprocal plot derived from the results to calculate the kₘ value.

FIG. 22. Overexpression of PLS3 converts PMA to a cell death agonist. HeLa-control, HeLa-PLS3, and HeLa-PLS3(F258V) cells were incubated with DMSO (black), 200 nM PMA (green) or the combination of 200 nM PMA and 200 nM Go6976 (red) for 2 hours. Cells were then fixed with ethanol for TUNEL assays to quantify apoptosis.

FIG. 23. Overexpression of mitochondrial targeted PKC-δ. (a) Annexin V-PE study of the apoptosis was performed in the HeLa-control, HeLa-PLS3 and HeLa-PLS3(F258V) transfected with the mitochondrial targeted PKC-δ construct or control vector. The percentages of apoptosis were indicated. (b) immunofluorescent staining was performed in the vector-transfected control HeLa cells with PKC-δ antibody for colocalization with MitoTracker Red. (c) HeLa cells were transfected with expression vector of the mitochondrial targeted PKC-δ. Similar staining was performed with PKC-δ and MitoTracker Red. PKC-δ antibody staining (left panels); MitoTracker Red (middle panels); overlay (right panels).

### DETAILED DESCRIPTION OF THE INVENTION

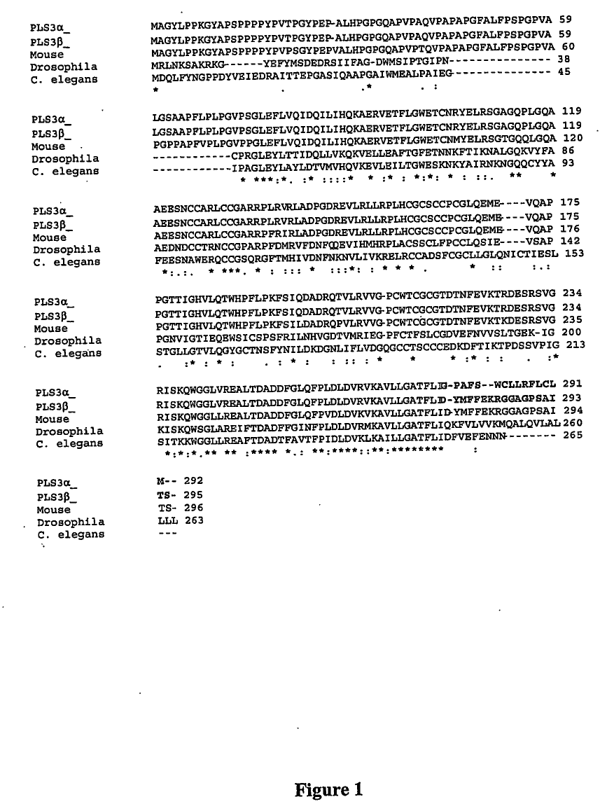

The present invention will be best understood by reference to the drawings and detailed description that follows. The following more detailed description of the gene, and methods of inducing apoptosis and creating apoptosis resistance using related genes of the present invention, as represented in FIGS. 1 through 23, is not intended to limit the scope of the invention, as claimed, but is merely representative of presently preferred embodiments of the invention.

As known to one of ordinary skill in the art, both nucleic acid sequences and amino acid sequences may be varied within the scope of the invention. More specifically, changes or mutations to either amino acid or nucleic acid sequences that are conservative are included within the scope of the invention. In the case of nucleic acids, such conservative substitutions may produce a protein having a sequence 85%, 90%, 95%, or even greater than 95% identical to the sequences disclosed herein and maintaining the function of the disclosed proteins. Such nucleic acid sequences and amino acid sequences are considered within the scope of the instant invention. In addition, mutations or deletions that have minor or no consequence on the function of the nucleic acid or amino acid are considered within the scope of the invention. Similarly, nucleic acids composed of naturally-occurring nucleotides, sugars and internucleotide (or “backbone”) linkages, as well as oligonucleotides having modified nucleotides, sugars, or backbone linkages, as well as oligonucleotides having mixed natural and modified nucleotides, sugars, and backbones or other non-naturally occurring portions that have similar function to naturally-occurring compounds are considered within the scope of the invention.

I. Phospholipid Scramblase 3 is a Downstream Effector of Cell Death Regulator Bcl-B

Bcl-B is the human orthologue of mouse BOO/Diva, a member of Bcl-2 family similar to avian NR13. Avian NR13 was originally identified as a v-src-activated gene (Gillet et al. 1995), and overexpression of NR13 protein protected Baf-3 cells from IL-3 withdrawal-induced apoptosis (Mangeney et al. 1996). NR13 plays a major role in regulation of chicken bursal apoptosis. NR13 protected a bursa-derived cell line, DT40, from serum-deprivation-induced apoptosis, and the level of NR13 correlated with bursal cell survival in vivo. Using a bursal transplantation model, it was demonstrated that one physiological function of NR13 is to protect the bursal stem cells from programmed elimination (Lee et al. 1999).

Searching for mammalian homologues of avian NR13, two groups identified mouse BOO/Diva through EST database analysis (Inohara et al. 1998; Song et al. 1999). It has a unique tissue distribution, expressed in all embryonic tissues but only in adult ovary. Protection from or promotion of cell death by BOO/Diva was cell-type dependent. BOO/Diva interacted with Apaf-1; however, the physiological significance of this interaction is unclear (Inohara et al. 1998; Song et al. 1999).

Bcl-B is highly conserved between mouse and human, and its anti- or pro-apoptotic effect is significantly weaker compared with Bcl-2 or Bcl-xL. Bcl-B interacts with Bcl-2, Bcl-xL and Bax, but not Bak (Ke et al. 2001). Bcl-B is not completely localized in mitochondria, but translocates to mitochondria upon induction of cell death by microtubule-interfering agents. Bcl-B gene was mapped in chromosome 15q21, and deletion was identified in some primary cervical cancers (Lee et al. 2001).

The biochemical functions of Bcl-2 family in mitochondria are far from clear. The crystal structure of Bcl-xL (Muchmore et al. 1996) suggested that Bcl-2 family members could form channels, and regulate the mitochondrial transmembrane potential (Δψ) and the release of cytochrome c upon pro-apoptotic stimulation (Shimizu et al. 1999; Vander Heiden and Thompson 1999; Shimizu et al. 2000; Shimizu and Tsujimoto 2000). The regulation of mitochondrial transmembrane potential (Δψ) is mediated through a transmembrane permeability pore complex (Shimizu et al. 1999). This multiprotein complex contains many components, including voltage-dependent anion channel (VDAC) and peripheral benzodiazepine receptor (PBR) on the outer membrane of mitochondria, adenine nucleotide translocator (ANT) on the inner membrane, and a matrix protein cyclophilin D (CphD).

Shimizu et al. used liposomes carrying VDAC to show that Bax and Bak accelerated the opening of VDAC, while Bcl-xL closed VDAC through direct interaction with VDAC. Using a BH4 domain peptide of Bcl-xL, they effectively prevented apoptotic cell death induced by etoposide treatment, indicating that BH4 domain of Bcl-xL was operative in this function (Shimizu et al. 2000). On the other hand, the BH3-only Bcl-2 family members induce release of cytochrome c without Δψ loss, and do not directly modulate VDAC activity (Shimizu and Tsujimoto 2000). These results demonstrated two different functions of the mitochondrial transmembrane pore complex, i.e. maintaining mitochondrial transmembrane potential (Δψ) and serving as a transmembrane protein channel, and they are regulated through different domains of Bcl-2 family members.

Cardiolipin has recently drawn attention as a mitochondrial phospholipid involved in apoptosis. This dimeric phospholipid contains two phosphatidyl molecules linked by a glycerol moiety, thus in total four fatty acyl chains and two phosphate groups (Hoch 1992). Cardiolipin is mainly located at the inner membranes of mitochondria (Krebs et al. 1979) and is required for oxidative phosphorylation and high energy electron transport (Jiang et al. 2000; Schlame et al. 2000). Cardiolipin interacts with cytochrome c, and peroxidation of cardiolipin dissociates cytochrome c from cardiolipin (Shidoji et al. 1999; Nomura et al. 2000).

Studies of cardiolipin were carried out with a cardiolipin-specific fluorescence dye NAO, nonyl-acridine orange (Garcia Fernandez et al. 2000). NAO has a fluorescence emission at 625 nm with 2:1 stoichiometric interaction to cardiolipin, and at 525 nm with 1:1 interaction. Measuring the amount of cardiolipin can be done with NAO by flow cytometry analysis (Garcia Fernandez et al. 2000). When cells become apoptotic, NAO binding decreases secondary to peroxidation of cardiolipin, a process that contributes to the release of cytochrome c (Shidoji et al. 1999). Another function of cardiolipin was recently identified as the mitochondrial target for tBid, which is cleaved from Bid upon apoptotic stimulation and induces activation of caspase 9 along with cytochrome c and Apaf-1 (Lutter et al. 2000).

The identification of a mitochondrial phospholipid scramblase 3 (PLS3) which interacts with Bcl-B is reported herein. It was found that PLS3 has a unique topology of crossing both outer and inner membranes of mitochondria, and PLS3 moves cardiolipin from the inner to the outer membranes of mitochondria. Overexpression of PLS3 significantly enhanced the pro-apoptotic effect of Bcl-B, and a PLS3 mutant prevents Bcl-B-induced cell death. These results indicate that PLS3 is a downstream effector of Bcl-B.

II. Phospholipid Scramblase 3 is a Member of the Scramblase Family that is Present in the Mitochondria

As previously noted, phospholipid scramblases are a family of enzymes responsible for bidirectional movement of phospholipids between two compartments (Bevers et al., 1999; Sims and Wiedmer, 2001). Four members of PLS family have been identified in the GenBank database (Wiedmer et al., 2000). PLS1 is the best-studied member and localizes in the plasma membrane. The remaining three members of the family are essentially uncharacterized.

During apoptosis, phosphatidylserine (PS) is translocated from the inner leaflet to the outer leaflet of the plasma membrane as a phagocytotic signal for macrophages (Fadok et al., 1992; Martin et al., 1995; Bratton et al., 1997; Zhao et al., 1998; Fadok et al., 2000; Fadok et al., 2001). It was suspected that the activity of PLS1 is related with PS flipping. However, cells from homozygous PLS1-deficient mice still have PS flipping in apoptosis presumably through complementation by other phospholipid translocase activities (Zhou et al., 2002). PLS1 can be phosphorylated at threonine and tyrosine by PKC-δ and c-abl kinases respectively (Frasch et al., 2000; Sun et al., 2001). However, it remains to be confirmed that the phosphorylation directly results in PLS1 activation (Zhao et al., 1998).

It is disclosed herein that another member of the PLS family, PLS3, is localized in the mitochondria. In mitochondria, PLS3 interacts with member of the Bcl-2 family, the human Boo/Diva (hBoo) and Bcl-2. Human Boo can either enhance or inhibit apoptosis similar to the mouse gene (Inohara et al., 1998; Song et al., 1999; Aouacheria et al., 2001; Ke et al., 2001; Lee, 2001). Mutations in hBoo were identified in human cervical cancer (Lee, 2001), but the significance and the biological function of hBoo remain unknown. Recently, the Boo/Diva deficient mice were generated by homologous recombination, but no obvious phenotype was observed (Russell et al., 2002).

Results and Discussion:

Cloning of PLS3 by interaction with hBoo.

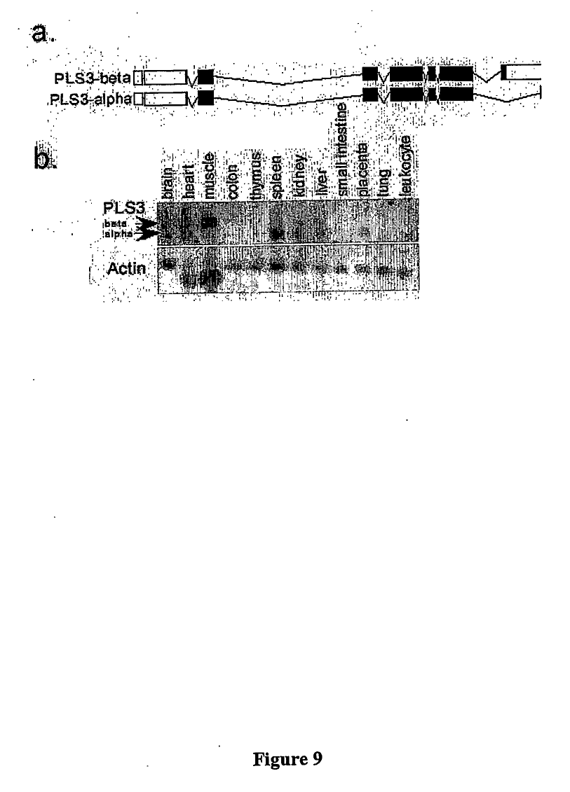

To understand the function of hBoo, its interacting protein was sought by yeast two-hybrid screening of a human liver cDNA library. Among the 17 clones that interacted specifically with hBoo but not with the control bait lamin C, three were identified as cDNA encoding amino acids 103-277 of phospholipid scramblase 3 (Wiedmer et al., 2000). The full-length cDNA clone was then identified (AW239215; accession no. 6571605, SEQ ID NO: 11) by searching the human EST database. The whole AW239215 clone was sequenced, and it was noted that it is a different isoform compared with the cDNA cloned from the yeast two-hybrid screen. The AW239215 clone contained 1722 nucleotides between the 5′ end cloning site and the poly A tail. Nucleotides 1017-1596 were missing in the cDNA clone previously obtained. The translated proteins differ only in the last 17 amino acids. The shorter form was designated PLS3-α and the longer form (AW239215) as PLS3-β. Using the Ensembl human genome database to look for the genomic structure of PLS3, it was found that PLS3 is located in human chromosome 17 at the telomeric side of p53 gene about 0.4 Mb in distance (Ensembl gene ID: ENSG00000174289), and the mouse orthologue is located at the corresponding mouse chromosome 11. The genomic structure of human PLS3 contains 8 exons and the last exon is alternatively spliced to generate PLS3α and PLS3β (FIG. 9a). Both splice junctions contain the required AG sequence right before the splice sites (the GT-AG rule). The two alternatively spliced messages are consistent with the two signals (2.4 and 1.8 kilonucleotides in size) in the northern blot (FIG. 9b). Blast search identified mouse, Drosophila and C. elegans homologues, indicating that this gene is highly conserved in evolution (FIG. 1).

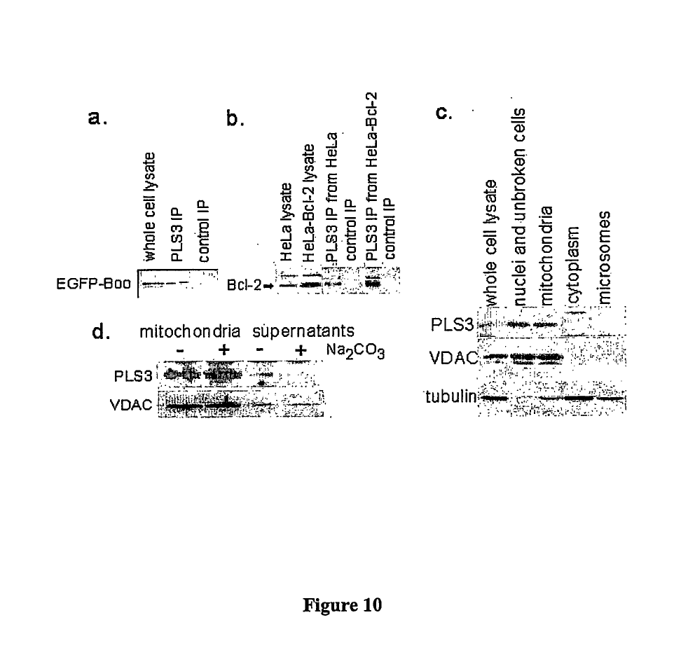

The in vivo interaction of PLS3 and hBoo was confirmed by immunoprecipitation (IP). HEK293 cells were transiently transfected with pEGFP-hBoo expression vector, and lysates were immunoprecipitated with affinity-purified PLS3 antibody. FIG. 10a shows that hBoo specifically co-precipitated with PLS3; while the control IP from HEK293 cells transfected with pEGFP vector failed to bring down EGFP by PLS3 antibody. This result indicates a physical interaction between PLS3 and hBoo. Since hBoo is a member of the Bcl-2 family, it was also studied whether PLS3 interacts with other members of the Bcl-2 family. The interaction with Bcl-2 was examined in both HeLa cells and HeLa cells overexpressing Bcl-2. The endogenous PLS3 from HeLa cells co-immunoprecipitated with Bcl-2, and more Bcl-2 was present in the PLS3 immunoprecipitates from HeLa-Bcl-2 cells (FIG. 10b). The control IPs with preimmune serum were negative. Similar study with Bcl-xL antibody was negative despite multiple attempts (data not shown), indicating that PLS3 binds Bcl-2 but not Bcl-xL.

Both PLS3-α and PLS3-β localize to mitochondria.

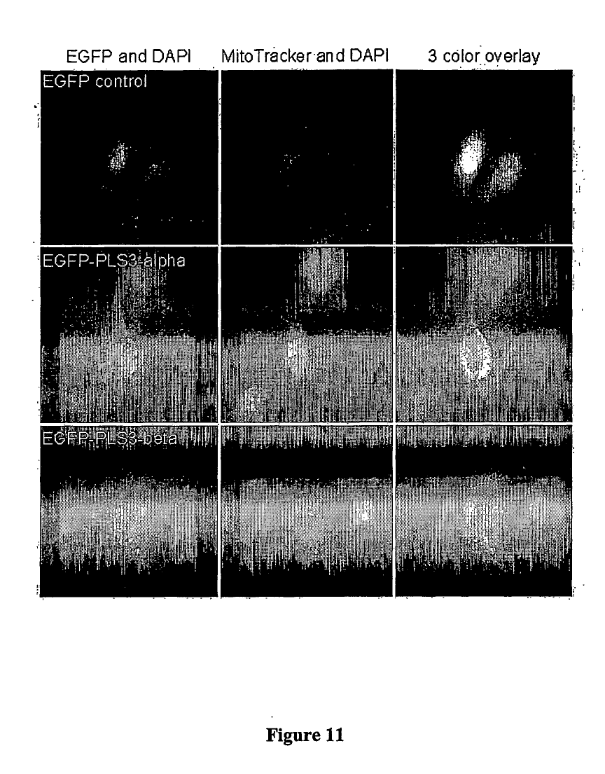

The interaction of PLS3 with members of the Bcl-2 family suggests that PLS3 may have a different localization from PLS1. Because our antibody could not detect native PLS protein by immunofluorescence staining, the subcellular localization of PLS3-α and PLS3-β was examined fused with EGFP at their N-terminus. Both EGFP-PLS3-α and EGFP-PLS3-β had a similar granular pattern. Cells were stained with MitoTracker Red, a mitochondrial dye, for co-localization. EGFP-PLS3 co-localized with MitoTracker Red (FIG. 11a), and cells transfected with control EGFP plasmid has a diffuse cytoplasmic pattern (FIG. 11b). This indicates that both PLS3-α and PLS3-β are in the mitochondria. Subcellular fractionation of mouse liver was then performed, and a Western blot confirmed that endogenous PLS3-α is predominantly present in mitochondria (FIG. 10c). The isolated mouse liver mitochondria were washed with Na₂CO₃ and confirmed that PLS3 can not be washed off with 0.1 M Na₂CO₃, and therefore is integrated in the mitochondria (FIG. 10d). No mitochondrial targeting sequence could be identified at the N-terminus of PLS3.

Proteins with mitochondrial targeting sequence are imported by the TIM/TOM complex into the matrix (Bauer et al., 2000). Several mitochondrial proteins, such as Bcl-2, VDAC, also lack any detectable mitochondrial targeting sequence since they do not depend on TIM/TOM complex for mitochondrial integration.

Topology of PLS3 in the Mitochondria

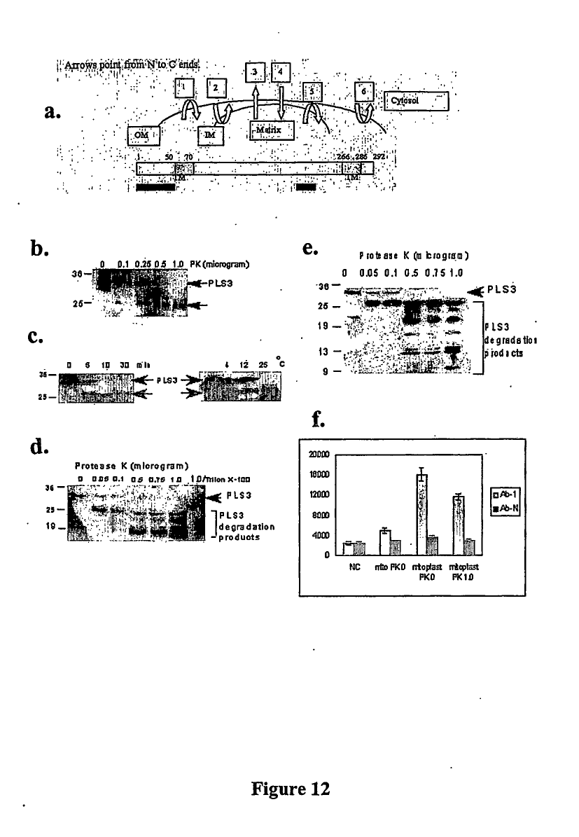

The topology of PLS3 in mitochondria was next investigated. Computer analysis (http://www.ch.embnet.org/software/TMPRED_form.html) of PLS3α predicted two transmembrane (TM) domains at amino acids 50-70 and 266-286, in contrast to one TM domain for PLS1. Given its two putative TM domains, PLS3 has six potential topologies with respect to the mitochondrial IM and OM. PLS3 could cross the OM twice (topologies 1, 6), the IM twice (topologies 2, 5), or both IM and OM once (topologies 3, 4) (FIG. 12a). Two different approaches were used to study the topology: proteinase K digestion and antibody epitope mapping. (Donzeau et al., 2000). It was anticipated that the proteinase K digestion of the intact mitochondria would generate a very small size PLS3 fragment recognized by Ab-N in topology 1, a slightly smaller fragment of PLS3 recognized by both Ab-1 and Ab-N in topologies 3, 4, and 6, and no PLS3 digestion in topologies 2 and 5 because they are buried inside of mitochondria.

Due to the concern that overexpressed protein may have aberrant topology, mitochondria isolated from mouse liver were used, which have abundant native PLS3 protein (Wiedmer et al., 2000). By incubating mitochondria in HEPES-mannitol buffer to preserve the mitochondrial OM, a dose-dependent proteinase K digestion was performed to cleave the portion that is outside of mitochondria, and analyzed the digested mitochondria with Ab-N and Ab-1 antibodies. Western analysis of the digested mitochondria revealed that the 30% D PLS3 protein was digested to a smaller fragment migrating at 25 kD (lower arrow, FIG. 12b). Similar results were obtained in a limited proteinase K digestion from 5 min to 30 min, or 4° C. to 25° C. (FIG. 12c). These results indicate that the PLS3 protein in intact mitochondria is accessible to proteinase K, and that a small portion of PLS3 is present outside of mitochondria. Topologies 1, 2, 5 could thus be eliminated.

Next, purified mitochondria were incubated in a low concentration HEPES buffer to generate mitoplasts by osmotic swelling and disruption of the OM. Using MAO and MDH, it was shown that this mitoplast preparation contained 76% of total MDH activity and 20% of total MAO activity, and the OM fraction contained 24% of MDH and 80% of MAO activity (Ragan, 1995). Although mitoplasts still contained about 20% of the residual OM (likely sticking to mitoplasts at the junctional zone), proteinase K could still have access to the intermembranous space through the holes generated by osmotic swelling, and is capable of digesting proteins at the intermembranous space (Donzeau et al., 2000). It was anticipated that digestion of the portion in the intermembranous space would generate a fragment of 70 amino acids (the N-terminal 50 amino acids plus the 20 amino acids of the TM domain) only recognized by Ab-N, but not Ab-1, in topology 3. In topologies 4 and 6, both epitopes recognized by Ab-1 and Ab-N would be digested, resulting in no fragments in Western blot.

Western analysis of proteinase K-treated mitoplasts revealed further cleavage of the 25 kD product to smaller fragments at sizes of 15-25 kD (FIG. 12d). The undigested OM fraction failed to show any PLS3 protein (FIG. 12d far right lane). These two findings eliminated topology 6, in which PLS3 should only be present in the OM. In Western analysis with Ab-N, a 9 kD band was detected by Ab-N at the highest concentration of proteinase K digestion (FIG. 12e). This is compatible with topology 3 with only the first 70 amino acids remaining after digestion of the intermembranous portion of PLS3. It was thus concluded that topology 3 is the actual orientation of PLS3 in mitochondria, with the N-terminus in the matrix and the C-terminus on the outer surface of mitochondria.

Epitope mapping was then performed to study the localization of the epitopes recognized by the anti-PLS3 antibodies discussed above. As shown in FIG. 12f, intact mitochondria failed to be recognized by either Ab-1 or Ab-N, suggesting that neither epitope is outside of the mitochondria. Mitoplasts were recognized by Ab-1, but not by Ab-N (FIG. 12f), indicating that the epitope between the two TM domains recognized by Ab-1 resides in the intermembranous space, and that the N-terminus epitope recognized by Ab-N is inside the mitoplasts. Limited proteinase K digestion of the mitoplasts decreased Ab-1 recognition, confirming that the epitope of Ab-1 is outside of mitoplasts (FIG. 12f). These results are also consistent with the topology 3 (FIG. 12a). Therefore, both proteinase digestion and epitope mapping support that PLS3 protein crosses both mitochondrial IM and OM with the N-terminus in the matrix and the C-terminus outside of mitochondria. This type of mitochondrial topology has been described in Tim23, a subunit of the mitochondrial pre-protein translocase complex (Donzeau et al., 2000). This pre-protein translocase complex is responsible for translocating the pre-proteins that are synthesized in the cytoplasm into mitochondria. Without being limited to any one theory, it is thought that PLS3 could be responsible for moving phospholipids into or out of mitochondria. Unfortunately, very few studies were done regarding the movement of phospholipids into mitochondria, although the bioactive lipids are well known to play roles in the signal transduction processes. It is not clear whether they translocate to mitochondria.

Another possible function of PLS3 is to move phospholipids between the inner and outer membranes of the mitochondria based on its topology of expanding through both IM and OM. The phospholipid composition between the inner and outer membranes of mitochondria is different (Parsons, 1967; Parsons and Yano, 1967). The most dramatic difference in phospholipids is the amount of cardiolipin, a mitochondrion-specific phospholipid. Cardiolipin is predominantly present in the inner membrane of mitochondria and plays a role in oxidative phosphorylation and ATP production. Without being limited to any one theory, maintaining the cardiolipin homeostasis appears to be important for the normal mitochondrial functions. It remains to be determined whether PLS3 plays any role in mitochondrial phospholipid homeostasis.

III. Phospholipid Scramblase 3 Controls Mitochondrial Structure, Function, and Apoptopic Response

Regulation of programmed cell death is central to development and neoplastic transformation. Dysregulation of apoptotic mediators results in tumor clonal evolution and resistance to chemotherapy (Ionov et al., 2000; Reed, 1999). Morphologically, apoptosis is characterized first by mitochondrial disintegration, followed by nuclear fragmentation, chromatin condensation and cytoplasmic membrane blebbing (Cory and Adams, 1998; Kroemer and Reed, 2000).

Mitochondria are the integrators of many apoptotic pathways. Several pro-apoptotic regulators, including Bax, Bad, Bid, Bim, p53, JNK, PKC-δ, nuclear receptor TR3 (Brenner and Kroemer, 2000), and the Peutz-Jegher gene product LKB1 (Karuman et al., 2001), translocate to the mitochondria during apoptosis. Among these factors, Bid is cleaved at the N-terminus to become tBid by activated caspase 8 before mitochondrial targeting (Luo et al., 1998). This targeting of tBid is mediated by N-myristoylation (Zha et al., 2000) and interaction with cardiolipin (Lutter et al., 2000). Activated tBid induces oligomerization of Bax and Bak to form cytochrome c channels during apoptosis (Korsmeyer et al., 2000; Wei et al., 2000; Wei et al., 2001). Without the presence of Bax or Bak, cytochrome c release or apoptosis is resistant to tBid (Wei et al., 2001).

During apoptosis, phosphatidylserine (PS) is translocated from the inner to the outer leaflet of the plasma membrane (Bratton et al., 1997; Fadok et al., 1992; Martin et al., 1995). Macrophages recognize PS in the outer membrane, and engulf apoptotic cells by phagocytosis (Fadok et al., 2000; Fadok et al., 2001). However, the regulation of the enzymes responsible for PS translocation remains elusive.

Phospholipid scramblases (PLS) are enzymes responsible for bi-directional movement of phospholipids (Bevers et al., 1999), and four PLS family members have been identified (Wiedmer et al., 2000). PLS1 is located in the plasma membrane and responsible for translocation of phospholipids between the inner and outer leaflets (Zhou et al., 1997). Although apoptotic PS translocation was unaltered in PLS1-deficient mice (Zhou et al., 2002), the role of PLS1 in apoptosis remains unclear given the presence of additional enzymes associated with plasma membrane phospholipid translocation such as aminophospholipid translocase (Bevers et al., 1999; Williamson et al., 2001; Zhao et al., 1998). PLS family members contain a conserved calcium-binding motif, and Zhou et al (Zhou et al., 1998) found that mutation of residues in this region of PLS1 completely eliminated enzymatic activity. PLS1 is phosphorylated at Thr-161 by PKC-δ, which translocates to the plasma membrane during apoptosis (Frasch et al., 2000); in addition, PLS1 is phosphorylated at Tyr-69/74 by c-abl kinase (Sun et al., 2001). The role of calcium binding and these various phosphorylation events in PLS 1 activation and regulation, however, remain to be determined.

A newly identified member of the scramblase family, designated PLS3, is localized to the mitochondrial rather than plasma membrane (Liu et al., 2003). However, little is known regarding the physiologic function of PLS3 in mitochondria. It is shown herein that PLS3, like PLS1, is phosphorylated by PKC-δ (Liu et al., 2003). A mitochondrial targeted PKC-δ dramatically enhanced susceptibility to apoptosis in cells overexpressing PLS3, suggesting that PLS3 is the direct mitochondrial effector of PKC-δ-induced apoptosis (Liu et al., 2003). It is reported herein that targeting PLS3 disrupts both mitochondrial structure and function, including translocation of cardiolipin from the mitochondrial inner membrane (IM) to the outer membrane (OM) during apoptosis.

Members of the protein kinase C (PKC) family of kinases play diverse roles in cell signaling, proliferation, apoptosis, and other cellular processes. PKC-δ is particularly associated with apoptosis. PKC-δ is present in the cytoplasm, and translocates to various organelles, including the nucleus, mitochondria and the plasma membrane upon apoptotic stimulation. One substrate of PKC-δ is phospholipid scramblase 1 (PLS1) in the plasma membrane. However, the consequence of PLS1 phosphorylation and how PLS1 contributes to apoptosis remain elusive. PKC-δ is activated during apoptosis by caspase-mediated cleavage to become catalytically active. This catalytic active fragment of PKC-δ translocates to the mitochondria to induce apoptotic events. Inhibition of PKC-δ blocks the disruption of the mitochondrial transmembrane potential. These results indicate that PKC-δ plays an important role in triggering apoptosis through a mitochondrion-dependent pathway. However, the substrate of PKC-δ in the mitochondria has previously been unidentified.

The function of phospholipid scramblase (PLS) is to translocate phospholipids bidirectionally between two compartments. PLS1 translocates phospholipids between the inner and outer leaflets of the plasma membrane, which is asymmetric in lipid composition. Although phosphatidylserine (PS) is translocated to the outer leaflet during apoptosis, it is still unclear whether PLS1 is responsible for this translocation. PLS3, which is present in the mitochondria, is discussed in detail herein. Overexpression of PLS3 in the HEK293 cells enhanced apoptosis induced by UV irradiation, and blocking PLS3 with a dominant negative mutant of PLS3 suppressed UV and etoposide-induced apoptosis (Dai, submitted). It is reported herein that PLS3 interacts with PKC-8, and is phosphorylated by PKC-δ with a very high affinity in vitro.

Cells overexpressing PLS3 became apoptotic upon treatment with phorbol ester and upon expression of mitochondrial targeted PKC-δ. These results indicate that PLS3 is the downstream target of PKC-δ.

### EXAMPLES

I. Phospholipid Scramblase 3 is a Downstream Effector of Cell Death Regulator Bcl-B

Cloning of PLS3 and Identification of Two Different Forms of Transcripts.

In order to understand the functions of Bcl-B, a yeast two-hybrid screen was performed to identify proteins that interact with Bcl-B. Since Bcl-B is most abundant in human liver (Lee et al. 2001), a yeast human liver MatchMaker cDNA library (Clontech, Palo Alto, Calif.) was used for this screening. After initial screening, seventeen true positive clones were identified. Three clones were identical to human phospholipid scramblase 3 (Wiedmer et al. 2000). Since the clone obtained lacked 5′ sequence, the full-length cDNA clone was identified (AW239215; GI:6571605, SEQ ID NO: 11) by searching the human EST database. The AW239215 clone was sequenced and it was noted that it is an alternatively spliced form compared with the cDNA cloned. The AW239215 clone contained 1722 nucleotides between the Eco RI cloning site and the poly A tail. Nucleotides 1017-1596 were missing in the cDNA clone obtained. The translated proteins differ only in the last 15 amino acids (FIG. 1). The shorter form was named PLS3α (SEQ ID NO: 2) and the longer form (AW239215) as PLS3β (SEQ ID NO: 4). The hydrophobicity of each was analyzed to search for transmembrane domains and found that PLS3α contains two transmembrane helices at amino acids 51-72 and 270-286. PLS3β contains one good candidate transmembrane helix at amino acids 51-72 and a weak hydrophobic helix at amino acids 262-282. Homologous proteins were identified in mouse, Drosophila and C. elegans, indicating that this gene is highly conserved in evolution. See FIG. 1. The coding region of PLS3 is complementary to the cDNA sequence of human non-receptor tyrosine kinase TNK1 (AF097738) at the 3′ end untranslated region. TNK1 is closely linked with the human tumor suppressor gene p53 and mapped to human chromosome 17p13 (Hoehn et al. 1996).

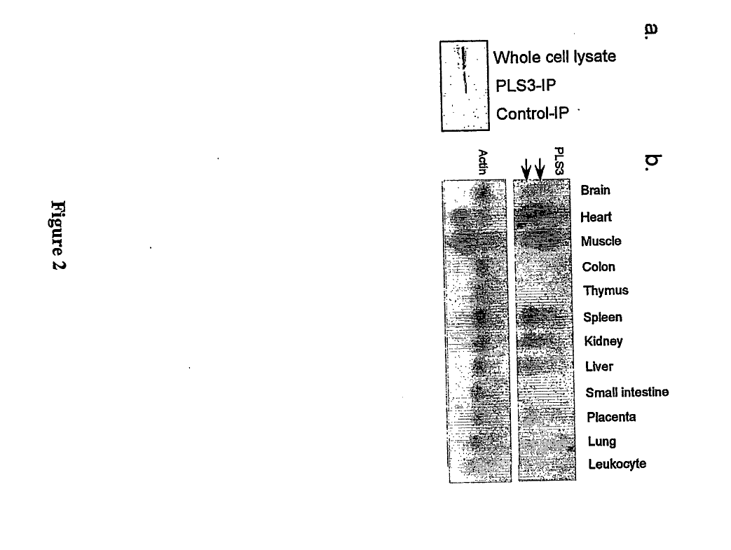

The in vivo interaction of PLS3 and Bcl-B was confirmed by immunoprecipitation (IP). 293T cells were transiently transfected with pEGFP-Bcl-B and pcDNA-PLS3α expression vectors, and harvested for immunoprecipitation with affinity-purified PLS3 antibody. Western analysis was then performed with GFP antibody, which revealed that EGFP-Bcl-B was present in PLS3 immunoprecipitates (FIG. 2a). This result demonstrated the physical interaction of PLS3 and Bcl-B proteins in cells.

Tissue Distribution of PLS3α and PLS3β

Using a Human 12-Lane Multiple Tissue Northern (MTN) blot (Clontech, Palo Alto, Calif.), northern analysis was performed to study human PLS3 expression. It was found that PLS3 was expressed in two forms as predicted from our identification of two alternative splice forms. PLS3α was a 1.8 K message that was abundantly expressed in human spleen, kidney, liver, placenta, and less abundantly in brain, heart, and peripheral blood leukocytes. PLS3β was a 2.4 K message highly expressed in skeletal muscle, heart, and small amounts in brain. See FIG. 2b. It has been have previously reported that Bcl-B was expressed in human liver, kidney and ovary (Lee et al. 2001). This finding indicates that PLS3 has other physiological functions or modulators independent of Bcl-B.

PLS3α and PLS3β were both localized in mitochondria, and an N-terminal deletion disrupted mitochondrial transmembrane potential.

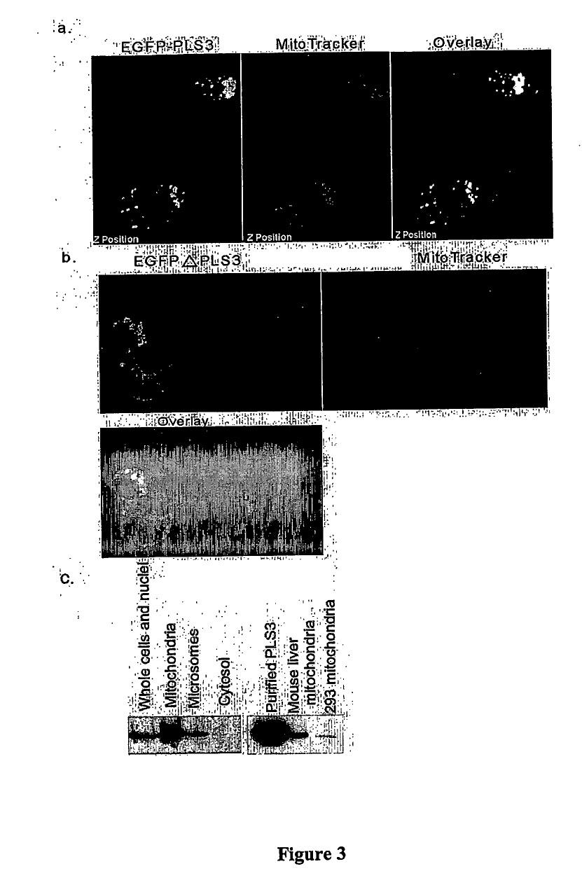

In order to investigate the subcellular localization of PLS3, PLS3α and PLS3 were fused with green fluorescent protein (GFP). The mitochondria of the transfected cells were stained with MitoTracker Red dye for co-localization. GFP fusion proteins of both PLS3α and PLS3β had identical patterns, and both were co-localized with MitoTracker dye. See FIG. 3a. This indicates that both PLS3α and PLS3β are in mitochondria. To ensure that GFP has no effect on localization, subcellular fractionation of mouse liver and cells transfected with pcDNA-PLS3α were performed for Western analysis. PLS3α was predominantly present in mitochondria, with a small amount in nuclei and microsomes, and was not detectable in cytosol. See FIG. 3c.

The accumulation of MitoTracker dye in mitochondria of PLS3α and PLS3β transfected cells indicated that they still had intact mitochondrial transmembrane potential. They also remained viable after G418 selection. A GFP fusion protein was made with an N-terminal deletion mutant, ΔPLS3α, which lacks the first 100 amino acids. Cells with GFP-ΔPLS3α expression were not labeled with MitoTracker Red, indicating that they could not accumulate MitoTracker dye, a process dependent on Δψ. See FIG. 3b. They eventually died after 7 days. This indicated that overexpression of GFP-ΔPLS3α disrupted mitochondrial Δψ and eventually caused apoptotic cell death, suggesting that the first 100 amino acids played a regulatory role in PLS3 functions.

Determination of PLS3 Topology

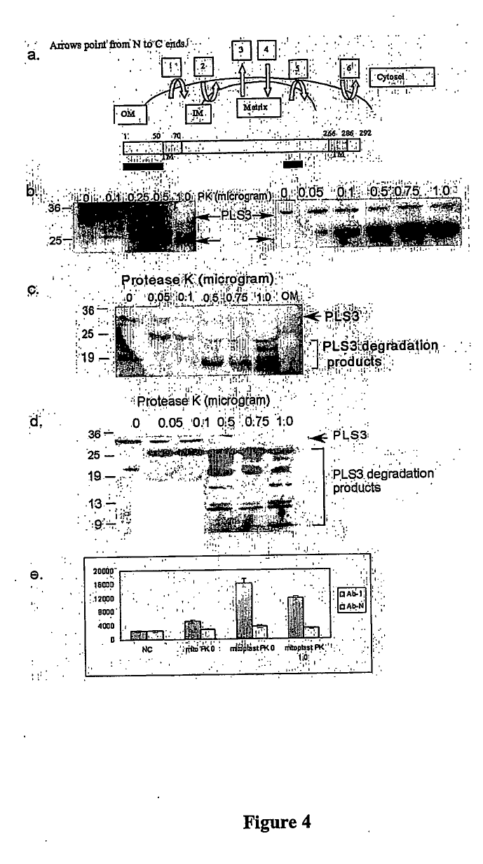

Since mitochondria have inner and outer membranes separating the intermembranous space and matrix, the topology of PLS3 in mitochondria was investigated to facilitate studying the function of PLS3. Rat liver was used to study the native PLS3α protein with the concern that overexpressed protein may not be localized in the appropriate position. By computer prediction, there are two putative TM domains (amino acids 50˜70 and 266˜286) in PLS3α protein, the isoform in liver, and therefore six potential topologies of PLS3 regarding its location at the inner and outer membranes of mitochondria. See FIG. 4a. PLS3 could be crossing the inner membrane twice (topology 1, 6), crossing the outer membrane twice (topology 2, 5), or crossing both inner and outer membrane once (topology 3, 4). Protease digestion and antibody epitope mapping were then used to identify the correct topology (Donzeau et al. 2000) (Hermann et al. 1998). Mitochondria were first purified with a sucrose gradient so that the mitochondria are at least 90% pure (Yuryev et al. 2000). By incubating mitochondria in HEPES-mannitol buffer to maintain the mitochondrial outer membrane intact, a dose-dependent limited protease K digestion was performed and analyzed the digested mitochondria with Ab-N or Ab-1 antibodies. These two antibodies recognize the N terminal 50-residue epitope or amino acids 219˜232 in the region between the two transmembrane domains, respectively. See FIG. 4a. In the Western analysis with Ab-N or Ab-1, the 30 kD PLS3 protein is digested to a 25 kD fragment. See FIG. 4b. Similar results were obtained in a limited protease K digestion from 5 min to 30 min or 4° C. to 25° C. (data not shown). These results suggested that the PLS3 protein is accessible to protease K in the intact mitochondria. In other words, PLS3 protein has a small portion present at outside of mitochondria. Topologies 1, 2, 5 could be eliminated based on these results.

Purified mitochondria were then incubated with a low concentration HEPES buffer to generate mitoplasts by osmotic swelling and disruption of the outer membranes. This allows protease K accessible to the intermembranous space of mitochondria, but not to the matrix. The cleavage products analyzed by Ab-1 Western blot revealed further cleavage of the 25 kD product to smaller fragments at sizes of 15-25 kD. Western analysis of the undigested outer membrane fraction failed to show any PLS3 protein (see FIG. 4c far right lane). These two findings eliminated possibility 6, which is expected to have PLS3 present in the outer membrane fraction. When the protease K cleavage products of mitoplasts were analyzed with Western blotting by Ab-N, it was observed at the highest concentration of protease K a smallest band at 9 kD which is still recognized by Ab-N (see FIG. 4d). This is compatible with topology 3 with only the first 70 amino acids left after digestion of the intermembranous portion of PLS3. Topology 3 was thus confirmed to be the correct orientation of PLS3 in mitochondria, with the N-terminus at the matrix side of mitochondria and the C-teminus at outside of the outer membrane of mitochondria.

Topology was also determined by antibody epitope mapping. Intact mitochondria and isolated mitoplasts were used to study the localization of the two epitopes recognized by two antibodies. Mitochondria or mitoplasts were used for incubation with either of the antibodies. After removing the excessive antibodies by washing, they were incubated with a secondary antibody conjugated with FITC for quantification of first antibody bound in pelleted mitochondria or mitoplasts. As showed in FIG. 4e, intact mitochondria failed to be recognized by both Ab-1 and Ab-N, suggesting that neither of the epitopes is outside of the mitochondria. On the other hand, mitoplasts were recognized by Ab-1, but not by Ab-N. This result indicates that the epitope between the two TM domains is at the intermembranous space of mitochondria and the N-terminus is inside of mitoplasts. When mitoplasts were treated with protease K, the antibody recognition by Ab-1 decreased, indicating that the epitope recognized by Ab-1 is sensitive to protease K and therefore outside of mitoplasts. All these results are consistent with the topology 3. Both protease digestion and epitope mapping studies thus lead to the conclusion that the PLS3 protein crosses both inner and outer membranes of mitochondria with the N-terminus at the matrix of mitochondria. This is the second protein reported to cross both mitochondrial inner and outer membranes in addition to Tim23, a subunit of mitochondrial preprotein translocase complex (Donzeau et al. 2000).

PLS3 protein disrupts mitochondrial transmembrane potential and induces cytochrome c release in vitro.

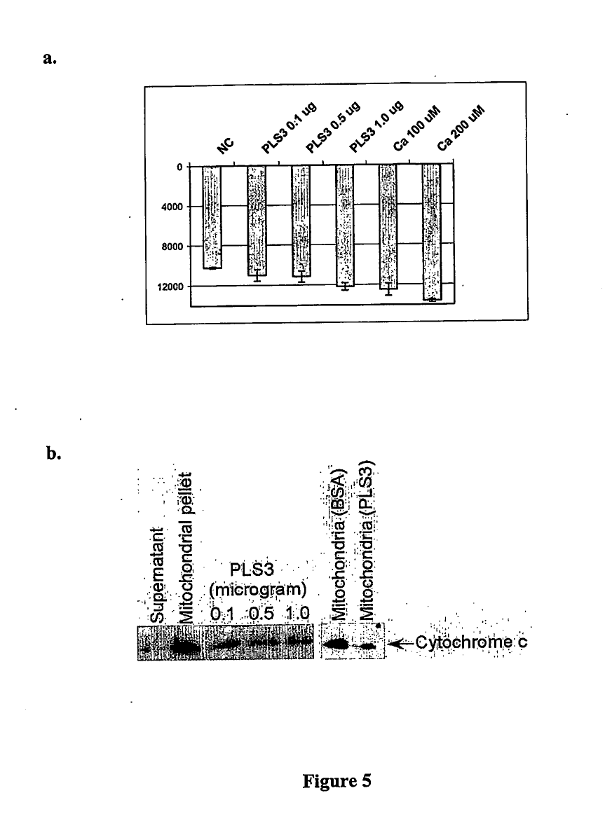

Using freshly isolated mouse liver mitochondria, an in vitro system was set up to study PLS3 function. Because PLS3α could not be overexpressed in bacteria, it was investigated whether purified PLS3β protein interferes with mitochondrial Δψ. Mouse liver mitochondria were incubated with PLS3β protein and then Δψ was measured with Rhodamine 123. The system was tested with calcium as a positive control, since calcium is known to induce disruption of Δψ (Duchen 2000). It was found that PLS3β protein induced disruption of Δψ in a dose dependent manner similar to calcium (FIG. 5a). The supernatant of PLS3β and mitochondria mixtures was then separated by centrifugation. Cytochrome c release was next analyzed with Western blotting. Cytochrome c was present in the supernatants of PLS3-treated mitochondria but not in buffer-treated mitochondria (FIG. 5b). This showed that exogenous PLS3 induced disruption of Δψ and release of cytochrome c in vitro. These results seem to be inconsistent to the in vivo overexpression of PLS3β, which did not induce loss of Δψ, and cells were viable with this overexpression. Without being limited to any one theory, it is thought that this could be due to a presence of inhibitors in cells, which prevents PLS3 from disruption of Δψ. The loss of Δψ and lethality of N-terminal deletion mutant indicate that this inhibitor may mediate its effect through this N-terminal portion. Another possibility is that PLS3 is post-translationally modified, such as phosphorylation in PLS1, to maintain PLS3 in an inactive state. The existence of two bands in PLS3 Western analysis may support this hypothesis. See FIGS. 2a and 4b.

PLS3 is a Cardiolipin Transferase In Vitro.

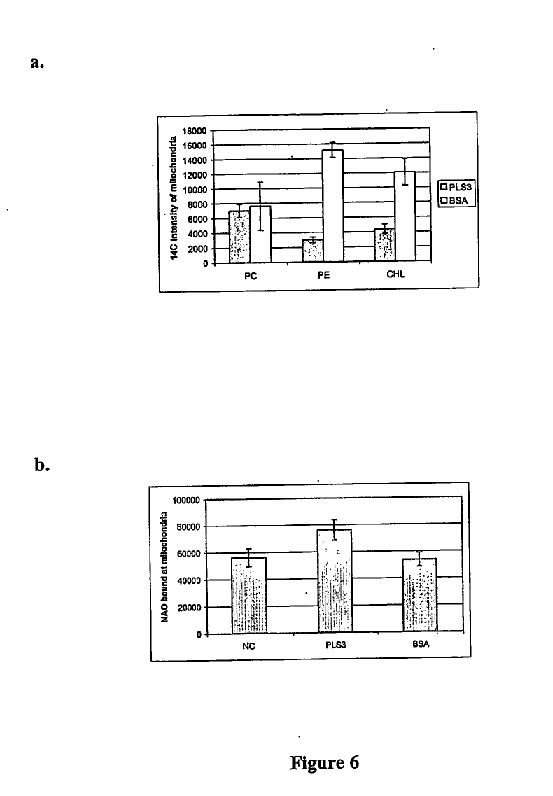

Since PLS3 has sequence homology with human plasma membrane phospholipid scramblase (PLS1) (Wiedmer et al. 2000), a phospholipid transfer assay was performed to determine the substrate specificity of PLS3. For this purpose, a purified PLS3β protein was used for an in vitro phospholipid transfer assay. Whether PLS3 had transferase activities for phosphatidylcholine (PC), phosphatidylethanolamine (PE) and cholesterol (C) using ¹⁴C-labeled phospholipids as substrates was first evaluated. When PLS3β protein was incubated with liposomes containing ¹⁴C-labeled PC and recipient mitochondria, the recipient mitochondria had no difference in activity compared with BSA-treated mitochondria (FIG. 6a). In contrast, studies with PE and cholesterol showed that PLS3β inhibited the intrinsic phospholipid transferase activities for PE and cholesterol (FIG. 6a). This finding could be related to the fact that PLS3 induced disruption of mitochondrial transmembrane potential (FIG. 5a), but the exact mechanism remains to be determined.

Cardiolipin was then investigated since cardiolipin is a mitochondrion-specific phospholipid and is potentially more critical in the process of apoptosis. Because radioactive cardiolipin is not commercially available, NAO was used to quantify cardiolipin in the recipient mitochondria. NAO binds cardiolipin specifically without interference by other phospholipids with a stoichiometry of 1 to 1 or 2 to 1. To achieve maximal NAO saturation, the recipient mitochondria were permeablized and fixed with formaldehyde. A dose titration of NAO was then performed. The red fluorescence achieved a plateau when the NAO concentrations were above 20 μM as reported (Garcia Fernandez et al. 2000). The concentration of 30 μM was thus chosen to determine the total amount of cardiolipin after in vitro cardiolipin transfer assay. It was confirmed that PLS3 increased the total amount of cardiolipin in recipient mitochondria by 35%, while mitochondria treated with buffer (NC) or bovine serum albumin (BSA) control had similar basal levels of cardiolipin (FIG. 6b). These results confirmed that PLS3 functions as a mitochondrial cardiolipin transferase.

PLS3 increases the percentage of cardiolipin at the outer membranes of mitochondria.

Once it was established that PLS3 is a mitochondrial cardiolipin transferase, it was investigated whether overexpression of PLS3 had any effect on total mitochondrial cardiolipin in 293 cells. Cells were transfected with pPLS3-IRES-EGFP or pIRES-EGFP for control and sorted for GFP positive cells. Western analysis confirmed the overexpression of PLS3 protein. Total amounts of cardiolipin were determined by NAO using the fluorescence intensity at 630 nm to represent cardiolipin amount. It was found that GFP positive cells from pPLS3-IRES-EGFP transfection had no difference in cardiolipin compared with the GFP positive cells from control pIRES-EGFP transfection (not shown). It was thus suspected that PLS3 translocates cardiolipin between two intracellular compartments, such as the inner and outer membranes of mitochondria. In this case, the total amount of cardiolipin remains unchanged with PLS3 overexpression.

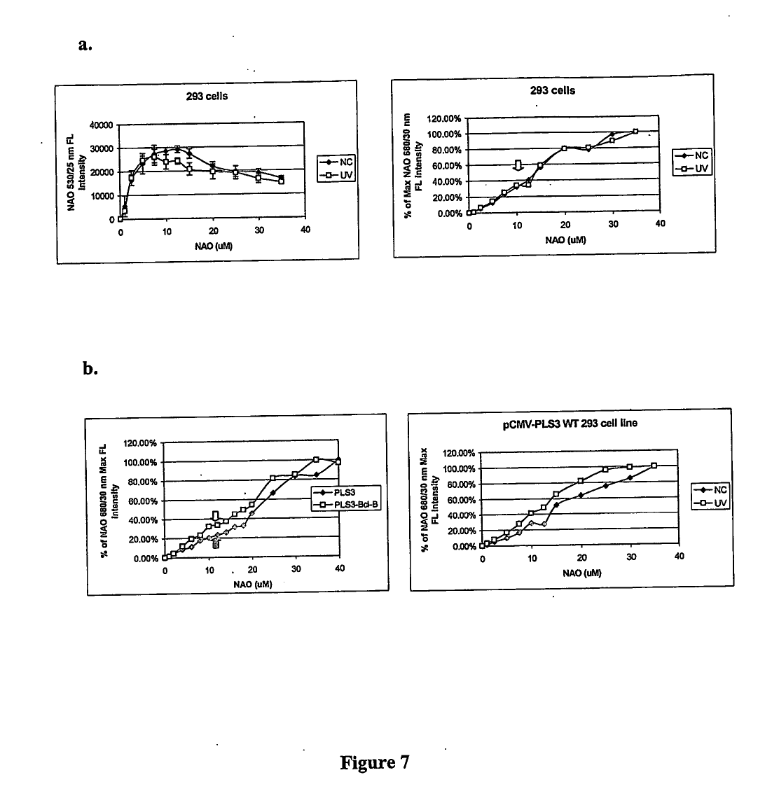

It is known that cardiolipin is mainly at the inner membrane of mitochondria with the ratio of cardiolipin between the outer and inner membranes about 3:21 or 12% at the outer membranes (Parsons 1967; Parsons and Yano 1967). The amount and redistribution of cardiolipin in the PLS3 overexpressing cells with or without UV irradiation-induced apoptosis was then investigated. 293 cells were first tested, and NAO dose titration was performed 10 hours after UV irradiation. In the curve of 530±25 nm, UV irradiation induced an expected decrease in NAO bound cardiolipin (FIG. 7a left), which is related with peroxidation of cardiolipin as reported (Shidoji et al. 1999). Fluorescence was then analyzed at 680±30 nm, which provides information about cardiolipin composition at the inner and outer membranes as described (Garcia Fernandez et al. 2000). By slowly increasing the NAO concentration, cardiolipin at the outer membrane of mitochondria becomes NAO saturated first, and then NAO enters mitochondria to interact with cardiolipin at the inner membrane. There is a shoulder after saturation of outer membrane, and the percentage of cardiolipin at the outer membrane could be represented by the percentage of cardiolipin at this shoulder versus total cardiolipin. The percentage of cardiolipin at the outer membrane of mitochondria in 293 cells is about 10% (solid arrowhead), which is barely visible. With UV irradiation, the shoulder increases to 30% (open arrowhead, FIG. 7a right). This means that UV irradiation increases the proportion of cardiolipin at the outer membrane of mitochondria.

Cells with PLS3 overexpression were then investigated. At the curve of 530±25 nm, there is an early rise of fluorescence up to 10 μM NAO, suggesting that cardiolipin at the outer membrane increases (FIG. 7b left). Using 680±30 nm to quantify the percentage, it was observed that the shoulder of non-irradiated cells is at 30% (solid arrowhead), which is higher than the 10% in normal 293 cells. After UV irradiation of PLS3 expressing cells, the shoulder further increases to about 50% (open arrowhead, FIG. 6b right). These results indicated that overexpression of PLS3 increases the proportion of cardiolipin at the outer membranes of mitochondria and UV irradiation further increases the percentage.

PLS3 Enhances the Apoptotic Effect of Bcl-B.

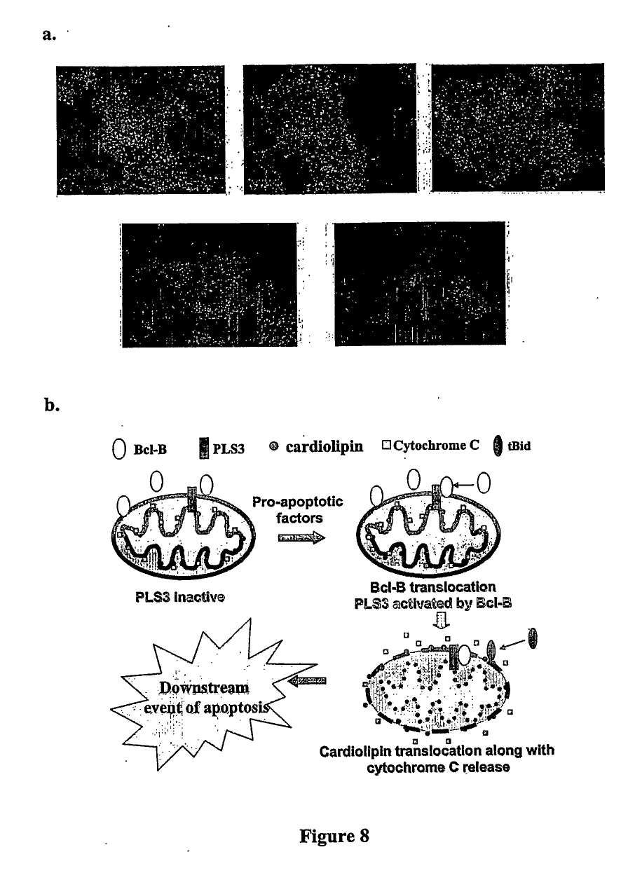

Bcl-B is a cell death agonist which translocates to mitochondria upon apoptotic stimulation (Lee et al. 2001). In this study, it was found that Bcl-B interacts with a mitochondrial enzyme, PLS3. Since the biochemical functions of most Bcl-2 family members are unclear, finding out whether Bcl-B functions through modulation of PLS3 activity and in determining the consequence of Bcl-B and PLS3 interaction was important to evaluate. Equal amounts of plasmids of either or both Bcl-B and PLS3 expression vectors were co-transfected into 293T cells. It was observed that cells transfected with pcDNA vector only or pcDNA-PLS3 plasmids had no difference in apoptosis compared with non-transfected cells. Cells transfected with pcDNA-Bcl-B has increased apoptosis as expected (Lee et al. 2001); while cells transfected with both pcDNA-Bcl-B and pcDNA-PLS3α developed significantly more apoptosis than Bcl-B alone (FIG. 8a). Using propidium iodide, the proportion of sub-G1 cells was quantified, or the apoptotic population, for cells without transfection or transfected with pcDNA control, pcDNA-PLS3α or pcDNA-Bcl-B as 1.4%, 2.8%, 3.8% and 9.8%, respectively. Cells transfected with both pcDNA-PLS3α and pcDNA-Bcl-B resulted in 15.4% apoptosis. Without being limited to any one theory, it appeared that this indicated that the cell death effect of Bcl-B was enhanced by co-expression of PLS3. In order to eliminate the possibility that difference in the amounts of transfected DNA contributed to the differences in apoptosis, sequential transfection was performed. A clone of GFP positive cells was obtained from a pPLS3-IRES-EGFP transfection and transfected this clone with pcDNA-Bcl-B. As a control, pcDNA-Bcl-B were transfected into GFP positive cells obtained from a pRES-EGFP transfection. Significantly more apoptotic cells were observed in pcDNA-Bcl-B transfected into PLS3 cells (not shown). This result confirmed that expression of PLS3α enhanced the death promoting effect of Bcl-B. Because Bcl-B physically interacts with PLS3, it was thought that Bcl-B may activate PLS3 through this interaction. Once PLS3 is activated, cardiolipin translocates in mitochondria and induces disruption of Δψ and cell death.

Activation of PLS3 by Bcl-B.

In order to prove that Bcl-B induces direct activation of PLS3, Bcl-B expression vectors were transfected into 293-PLS3 cells and the percentage of cardiolipin was measured at the outer membrane of mitochondria. Using the same methods as used in FIG. 7, it was observed that Bcl-B expression in 293-PLS3 cells shifted the curve of red fluorescence upward as was seen in UV irradiation of 293-PLS3 cells. The percentage of the shoulder, which represents the percentage of cardiolipin at the outer membrane, increase from 30% in 293-PLS3 cells to 50% in Bcl-B expressers, which is similar to that of UV irradiation (FIG. 7c). This result supports that Bcl-B induces activation of PLS3.

An enzymatically inactive mutant of PLS3 was constructed to determine whether this mutant prevents Bcl-B-induced apoptosis. By comparing the sequences of PLS1 and PLS3, a conserved amino acid Phe258 was identified which is located at a conserved calcium-binding motif of PLS3. Mutation of the corresponding Phe in PLS1 completely abolished the activity of PLS1 (Zhou et al. 1998). Phe258 was mutated to valine and a pCMV-PLS3(F258V) expression vector was constructed. 293 cells were then transfected with this vector and a stable transfected clone was obtained which has an obvious phenotype of slow growing. Apoptosis was analyzed in pCMV-PLS3(F258V) cells transfected with pBcl-B-IRES-EGFP to compare with 293-pCMV control cells transfected with the same Bcl-B expression vector. Cell death was detected in 14% of GFP positive cells in 293-pCMV control cells after transfection. However, only 7.5% of GFP positive cells in PLS3(F258V) expressors were apoptotic. This result indicates that PLS3(F258V) inhibits the cell death effect of Bcl-B, and confirms that PLS3 is a downstream effector of Bcl-B.

Discussion

Bcl-B-specific functions were then investigated by identifying PLS3 that interacts with Bcl-B. In contrast to PLS1, PLS3 is localized in mitochondria and has the substrate specificity to cardiolipin. To understand where PLS3 moves cardiolipin to, the topology of PLS3 in mitochondria was determined. It was found that PLS3 has a rare topology by crossing both inner and outer membranes of mitochondria. This type of mitochondrial topology has only been described in Tim23, a protein operative in mitochondrial preprotein translocation (Donzeau et al. 2000). Tim23 is responsible for translocating pre-protein into mitochondria; while PLS3 is likely responsible for cardiolipin translocation between inner and outer membranes. These studies appeared to confirm this theory. This study demonstrated a cardiolipin shift from the inner membrane to outer membrane by PLS3 during the process of apoptosis. This is reminiscent of PLS1-related PS translocation in the plasma membrane, and will facilitate more investigations in mitochondrial phospholipid changes during apoptosis.

The consequences of cardiolipin redistribution in mitochondria were also studied. Cardiolipin is associated with the major proteins of oxidative phosphorylation, such as ATP synthase, respiratory complex I, II and IV, as well as the carrier protein for phosphate and adenine nucleotides (Schlame et al. 2000). A yeast mutant lacking cardiolipin synthase, the critical enzyme for making cardiolipin, was viable; but moderately deficient in mitochondrial energy transforming machinery at 25° C. (Jiang et al. 2000). When the temperature increased to 37-40° C., respiration was completely uncoupled from phosphorylation, and mitochondrial transmembrane potential was disrupted (Koshlin and Greenberg 2000). This confirmed the importance of cardiolipin in critical mitochondrial functions.

The fate of cardiolipin during the process of apoptosis has just begun to be addressed. Cardiolipin undergoes peroxidation during apoptosis, and this peroxidation leads to dissociation of cytochrome c from cardiolipin (Shidoji et al. 1999; Nomura et al. 2000). Cytochrome c release and apoptosis were inhibited by overexpression of mitochondrial hydroperoxide glutathione peroxidase, which prevented cardiolipin peroxidation (Nomura et al. 2000). Although it is unclear how peroxidation of cardiolipin leads to changes in oxidative phosphorylation and mitochondrial dysfunction, failure of cytochrome c binding with peroxidized cardiolipin will at least partially contribute to the release of cytochrome c from mitochondria. Otherwise, cytochrome c will still be sequestered in the intermembranous space by cardiolipin even the putative cytochrome c channels are wide open.

When cardiolipin is translocated from the inner membrane to the outer membrane of mitochondria, the enzymes and cofactors responsible for the oxidative phosphorylation will be disturbed. This can directly lead to a reduction in oxidative phosphorylation. Harris et al. provided evidences that Bax induces its death effect by reducing the oxidative phosphorylation and the Bax toxicity is reduced in yeast strains deficient in oxidative phosphorylation (Harris et al. 2000). On the other hand, Bcl-xL promotes efficient ADT-ATP exchange to allow mitochondria to adapt to change in metabolic demand to prevent apoptosis (Vander Heiden et al. 1999). These findings support the importance of oxidative phosphorylation in mitochondria in the process of apoptosis. Redistribution of cardiolipin during apoptosis will thus have a major impact in mitochondrial function.

Recently, Lutter et al. identified cardiolipin as the target of tBid during the process of apoptosis (Lutter et al. 2000). Because very little cardiolipin is normally present at the outer membrane of mitochondria, the translocated cardiolipin by PLS3 provides the required tBid receptor at the outer membrane during apoptosis. If PLS3 plays such a key role in this cardiolipin translocation, inhibition of PLS3 activity with a dominant negative mutant will be able to prevent apoptosis by a variety of death signals. This appears likely to be the case. Overexpression of a mutant PLS3(F258V) also prevents apoptosis-induced by UV irradiation in addition to Bcl-B expression (manuscript in preparation).

It was observed that the PLS3 protein disrupted Δψ and induced cytochrome c release in vitro. However, overexpression of PLS3 did not cause cell death or disrupt Δψ. One possibility is that PLS3 is post-translationally modified, such as phosphorylation, and rendered inactive in mitochondria. When cells undergo apoptosis, PLS3 becomes activated. The activated PLS3 subsequently moves cardiolipin in mitochondria and induces mitochondrial apoptotic change. The pro-apoptotic effect of N-terminal-deletion mutant of PLS3 is similar to the in vitro results of PLS3 in disrupting Δψ. This suggests that the regulatory effect is mediated through the N-terminal portion of PLS3, perhaps through interaction with an inhibitor in normal mitochondria. Phosphorylation of PLS3 may affect the interaction between PLS3 and the putative inhibitor. According to our topology result, the N-terminus is located at inner membrane and matrix, which is not accessible by factors from cytosol. The putative inhibitor is thus likely a mitochondrial protein present in mitoplasts.

From the point of view of a possible role in cell death regulator, PLS3 also needs to be in an inactive state in normal cells and becomes activated upon induction of apoptosis. Otherwise, cells would not be able to keep cardiolipin mainly in the inner membrane, and cells would spontaneously lose mitochondrial integrity and undergo apoptosis. This is similar to PLS1, which is also inactive in normal cells and become activated by PKC-δ during apoptosis (Frasch et al. 2000). Investigation of the mechanism of PLS3 activation will be a crucial study in regulation of apoptosis. One known mechanism is through interaction of Bcl-B. This idea is supported by our findings that PLS3 and Bcl-B interact physically, and the combination of Bcl-B and PLS3 greatly enhanced cell death compared with either PLS3 or Bcl-B alone. Furthermore, both UV-irradiation and Bcl-B expression induce an increase in the percentage of cardiolipin at the outer membrane of mitochondria. This strongly supports that PLS3 can be activated by Bcl-B in triggering cell death.

According to these observations, a model as shown in FIG. 8b is proposed. Apoptotic stimulation or Bcl-B translocation induces the activation of PLS3, which translocates cardiolipin to the outer membranes of mitochondria. This results in at least two consequences. One is to disturb mitochondrial oxidative phosphorylation and poor energy production, and the other is to help carrying cytochrome c to the outside of mitochondria for subsequent caspase activation. The translocated cardiolipin also helps to recruit tBid to mitochondria, which is one of the most powerful death factors. The combination of all these events eventually leads to total mitochondrial breakdown and the eventual cell death.

Experimental Procedures

I. Identification of PLS3 cDNA and Two Alternative Transcripts.

A complete system of human liver cDNA MatchMaker yeast two-hybrid library was purchased from Clontech (Palo Alto, Calif.). Bcl-B was cloned in-frame into the pAS2 vector at NcoI and EcoRI sites. The pAS2-Bcl-B plasmid was transformed into yeast strain PJ69-2A for mating with the pretransformed library, which was constructed in the Y187 yeast strain. The mated yeast culture was selected with drop-out media lacking adenosine, histidine, leucine and tryptophan (DO-4 media) as described in the company protocol. Positive clones were confirmed with B-gal colony-lift assay. All positive clones were grown in liquid culture and plasmids were isolated. They were re-confirmed by growth in DO-4 media after co-transformation with pAS2-Bcl-B into the PJ69-2A strain. They were considered false positive if PJ69-2A grew in selection media after transformed with plasmids in combination with the pAS2-lamin C control vector. The final confirmed clones were sequenced and identities were obtained through Blast search.

Construction of PLS3 Expression Vectors.

PLS3α, PLS3β and ΔPLS3 were inserted in-frame into the SmaI site of pEGFP-C1 vector (Clontech, Palo Alto, Calif.). PLS3α was also cloned into Hind m and Bam HI sites of pIRES-EGFP and pcDNA3.1 vector (Invitrogen, Carlsbad, Calif.) for overexpression from a CMV promoter. In order to overexpress PLS3β protein in bacteria, PLS3β was cloned in frame into pGEX6P3 vector (Amersham-Pharmacia, Piscataway, N.J.) at an EcoRI site and expressed the glutathione-S-transferase-PLS3 fusion protein in BL21 competent cells for affinity purification. PLS3β protein was cleaved from GST fusion protein by precision protease according to the supplier's protocol (Amersham-Pharmacia Biotech. Inc. Piscataway, N.J.).

Northern Analysis and Western Analysis.

The human 12-Lane MTN blots were purchased from Clontech (Palo Alto, Calif.). The PLS3 cDNA was labeled with α³²P-dCTP (NEN) by random priming and then hybridized with blots according to the company protocol. PLS3 antibody was raised in two rabbits against a peptide corresponding to amino acids 219-232 of PLS3 with the sequence CDTNFEVKTRDESRS (SEQ ID NO: 8). The peptides were conjugated to beads by EDC/Diaminodipropylamine Immobilization kit (Pierce, Rockford, Ill.) to prepare an affinity column. Antibody was purified by the affinity column according to the company protocol. Western analysis was performed by standard procedure with PLS3 antibody at 1:100 dilution of affinity-purified primary antibody and 1:1000 of goat anti-rabbit secondary antibody conjugated with horseradish peroxidase (Amersham-Pharmacia Biotech. Inc. Piscataway, N.J.). The blots were developed with chemiluminescence for autoradiography (Pierce, Rockford, Ill.).

Mitochondria and Mitoplasts Preparation.

Mitochondria were isolated by differential centrifugation. In brief, cells were treated with a buffer containing 300 mM sucrose, 10 mM Tris (pH 7.5), 5 mM EDTA, and 1 mM PMSF protease inhibitor for 5 min on ice. Cells were broken by passage through 25 G needle for 10 strokes. Nuclei and unbroken cells were collected by centrifugation at 1000×g for 5 minutes. Mitochondria were then collected by centrifugation at 10,000×g for 10 minutes. Microsomal fractions were pelleted by ultracentrifugation at 30,000×g for 1 hour. The supernatants of the final centrifugation were saved for cytosolic fractions. For further purification of mitochondria for topology study, crude mitochondrial fraction was separated by a sucrose gradient at 1-2 M in mitochondrial isolation buffer. Mitoplasts were prepared by osmotic disruption of outer membrane with 10 mM HEPES/KOH, pH 7.4. After incubation on ice for 20 min and stirring to remove outer membranes, mitoplasts were collected by 20,000×g for 15 min (Hermann et al. 1998).

In vitro measurement of mitochondrial transmembrane potential and cytochrome c release.

Purified mouse liver mitochondria were quantified by the amount of total protein. Before analysis of transmembrane potential, mitochondria were further washed with the isolation buffer without EDTA. For each in vitro assay, 100 μg of mitochondria were incubated with 200 nM Rhodamine 123 (Molecular Probe, Eugene, Oreg.) and fluorescent intensities at 585 nm were determined by FL600 fluorescence microplate reader (Bio-Tek Instrument, Inc.). Mitochondria were treated with 100 nM calcium as positive control or different amount of purified PLS3 protein for 10 min before the assay. The mixture of mitochondria and PLS3 protein was centrifuged at 10,000×g for 10 min to separate supernatants and mitochondrial pellets. The supernatants were subsequently analyzed by Western for cytochrome c.

Phospholipid Transfer Assay.

Phospholipid transfer assay was modified from Koumanov et al. (Koumanov and Infante 1986). In brief, phospholipids were dissolved in chloroform, mixed and evaporated to dryness with a stream of nitrogen. Dried phospholipids were resuspended with the same buffer used for isolating mitochondria and sonicated in water bath for 5 min. Liposomes, which contained 280 nmole cold phospholipids and 1 μl ¹⁴C-labeled phospholipids (NEN) for each reaction, were mixed with freshly-prepared mouse liver mitochondria along with purified PLS3β or bovine serum albumin (BSA) as control, for 20 minutes at 37° C. Mitochondria were then washed five times with mitochondrial preparation buffer to remove free liposomes and counted by scintillation counter for ¹⁴C activity. For cardiolipin transfer assays, total amounts of cardiolipin were quantified by specific fluorescence with NAO dye. NAO (30 μM) was added to recipient mitochondria, and fluorescence intensity was measured at 590±35 nm by fluorescence microplate reader.

Flow Cytometry Analysis.

Nonyl acridine orange (NAO) dye, [10-N-nonyl-3,6-bis (dimethylamino) acrydine], was purchased from Molecular Probe Inc. (Eugene, Oreg.). Cells were fixed with 1% formaldehyde for 15 minutes at room temperature and washed twice with cold phosphate buffered saline (PBS) before staining with 30 μM NAO. Cells were fixed with 95% of alcohol at 4° C. overnight before staining with 50 μM of propidium iodide for cell cycle analysis. AnnexinV-PE was used for apoptosis assay according to company protocol (PharMingen). Flow cytometry analyses were performed with FACScan cytometer (Becton Dickinson, San Jose, Calif.).

II. Phospholipid Scramblase 3 is a Member of the Scramblase Family that is Present in the Mitochondria

Identification of PLS3α

A MatchMaker yeast two-hybrid kit was purchased from Clontech (Palo Alto, Calif.). The cDNA of hBoo was inserted in-frame into the pAS2 vector and transformed into yeast strain PJ69-2A for mating with the Y187 pretransformed human liver cDNA library. The mated diploid yeast was selected with media lacking adenosine, histidine, leucine and tryptophan (DO-4 media). Positive clones were confirmed with β-galactosidase assay. Clones were re-confined by growing in DO-4 media after co-transformation with pAS2-hBoo into the PJ69-2A strain. The plasmid pAS2-lamin C was used to rule out non-specific interaction. The final confirmed clones were sequenced and identities were obtained through the BLAST search. The cDNA of PLS3 was inserted into pEGFP-C1 vector (Clontech) and pcDNA3.1 vectors (Invitrogen) for mammalian expression.

Western Analysis

PLS3 antibodies, Ab-1 and Ab-N, were raised in rabbits with a peptide corresponding to amino acids 219-232 of PLS3 (CDTNFEVKTRDESRS, SEQ ID NO: 8) and recombinant PLS3(amino acids 1-50), respectively. Antibodies were purified by the corresponding affinity columns. Western blot analysis was performed by standard procedures with PLS3 antibodies at 1:250 dilution and developed with chemiluminescence.

Subcellular fractionation and preparation of mitochondria and mitoplasts.

Mitochondria were isolated by differential centrifugation. In brief, mouse liver was incubated in mitochondrial isolation buffer containing 300 mM sucrose, 10 mM Tris-HCl (pH 7.5), 5 mM EDTA, 0.1% BSA and 1 mM PMSF for 5 min on ice. Cells were disrupted by douncing 10 strokes, and centrifuged at 1000×g for 5 min, 10,000×g for 10 min, and 30,000×g for 60 min, for collection of intact cells/nuclei, crude mitochondria and microsomes, respectively. The final supernatants were used as cytoplasms. To further purify mitochondria for topology study, crude mitochondria were loaded on a sucrose gradient of 1-2 M in the mitochondrial isolation buffer, and centrifuged at 100,000×g for 90 min in a table-top ultracentrifuge (Beckman OptimaTMMax). Mitoplasts were prepared by osmotic disruption of the mitochondrial outer membrane (OM) with 10 mM HEPES/KOH (pH 7.4), and centrifuged at 20,000×g for 15 min. To determine the purity of separation, we measured the activities of monoamine oxidase (MAO) and malate dehydrogenase (MDH), as OM and inner membrane (IM) markers (Ragan, 1995). The proteinase K digestion was performed as described (Donzeau et al., 2000).

Epitope Mapping of PLS3

Mitochondria or mitoplasts (200 μg) were incubated with Ab-1 or Ab-N at 1:100 for 30 min, and then with secondary antibody conjugated with fluorescein isothiocyanate (FITC) at 1:100 for 30 min. The fluorescent intensity of the pellet-bound FITC was measured by FL600 fluorescence microplate reader.

III. Targeting Phospholipid Scramblase 3 Disrupts Mitochondrial Structure and Function

Results

Both wild-type and mutant PLS3 localize to mitochondria

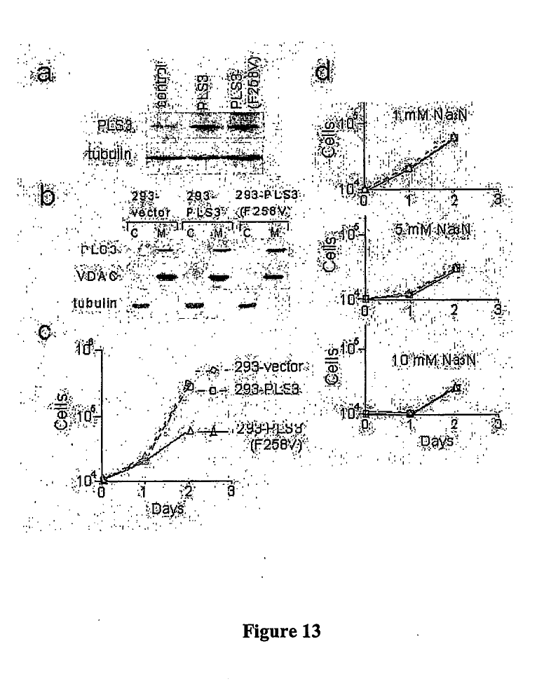

To investigate its function in mitochondria, we targeted PLS3 by mutating its calcium-binding motif that is highly conserved among PLS family members (Zhou et al., 1998). This approach was successfully employed by Zhou et al (Zhou et al., 1998), who found that mutation of Phe281 in PLS1 abolished function. By site-directed mutagenesis, we converted the corresponding Phe258 in PLS3 to valine, and generated stable transfectants of the mutant PLS3(F258V) in HEK293 cells. Transfectants of control (293-vector) and wild-type PLS3 (293-PLS3) were similarly prepared. Whole cell lysates of the G418-resistant clones were examined by Western blotting. Endogenous PLS3 could be detected in control whole cell lysates, and transfectants expressing the wild-type or mutant PLS3 demonstrated increased PLS3 levels (FIG. 13a). Subcellular fractionation indicated that the PLS3(F258V) protein localizes to the mitochondria, similar to the wild-type PLS3 (FIG. 13b). The integrity of cytosolic and mitochondrial fractions was confirmed by blotting for tubulin and voltage dependent anion channel (VDAC), respectively (FIG. 13b). Similar transfectants were established in HeLa cells (not shown) for additional experiments described below.

Slow Growth of Cells Expressing PLS3(F258V) Mutant

293-vector, 293-PLS3, and 293-PLS3(F258V) cells were cultured over a 3-day period and serial cell counts were performed to monitor cell proliferation. The growth rate of 293-PLS3 cells was comparable to that of 293-vector cells, but 293-PLS3(F258V) cells grew at a slower rate (FIG. 13c), indicating that the PLS3(F258V) mutant interferes with cell growth. Cell cycle analysis revealed that the slow growth did not result from spontaneous G1 or G2/M arrest (not shown). Next, we incubated these cells in the presence of Na₃N, an uncoupler of oxidative phosphorylation (Simbeni et al., 1990), and compared their growth. The same slow growth rate was observed at three different Na₃N concentrations, as the slope of all the growth curves was similar to that of the 293-PLS3(F258V) cells without Na₃N (FIG. 13d).

Targeting PLS3 Decreases Mitochondrial Mass, Transmembrane Potential, and Oxidative State

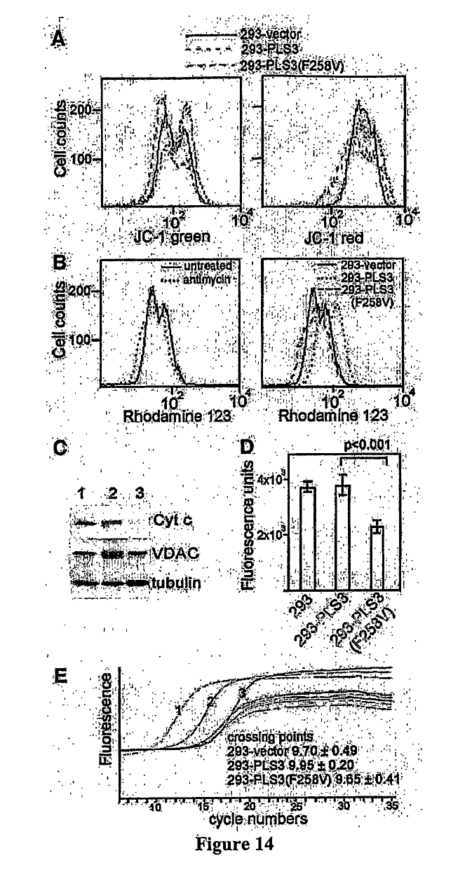

Because PLS3 localized to mitochondria and the growth of 293-PLS3(F258V) cells was not affected by uncoupling of oxidative phosphorylation, we suspected that the mitochondria might be defective in PLS3-targeted cells. First, we analyzed the mitochondrial mass and membrane potential using JC-1 dye and flow cytometry. There were two peaks in JC-1 green fluorescence, corresponding to the mitochondrial mass (Camilleri-Broet et al., 1998). The low intensity peak was predominant in 293-vector cells and 293-PLS3(F258V) cells, while the higher intensity peak was more prominent in 293-PLS3 cells (FIG. 14a, left). Analysis of JC-1 red fluorescence, corresponding to the mitochondrial transmembrane potential (Camilleri-Broet et al., 1998), revealed a relative right shift in 293-PLS3 cells and left-shift in 293-PLS3(F258V) cells (FIG. 14a, right). Thus, while over-expression of PLS3 was associated with increased mitochondrial mass and transmembrane potential, expression of PLS3(F258V) was associated with decreased mitochondrial mass and transmembrane potential.

Next, we measured the oxidative state of these cells using reduced Rosamine and flow cytometry. Over-expression of PLS3 was associated with a right-shift, indicating an increased oxidative state, compared to control cells (FIG. 14b). By contrast, expression of PLS3(F258V) was associated with a left-shift, or decreased oxidative state (FIG. 14b). The relatively decreased oxidative state of 293-PLS3(F258V) cells is consistent with the lack of inhibitory effect of Na₃N on growth described above.

Finally, we quantitated in these cells several mitochondrial markers that relate to oxidative phosphorylation. As shown in FIG. 14c, levels of cytochrome c were dramatically reduced in 293-PLS3(F258V) cells compared to 293-vector and 293-PLS3 cells. By contrast, levels of VDAC were unaffected in 293-PLS3(F258V) cells and slightly increased in 293-PLS3 cells (FIG. 14c). Staining for cytosolic tubulin served as a loading control (FIG. 14c). Given the interaction of cytochrome c with mitochondrion-specific cardiolipin(Shidoji et al., 1999), we determined the relative amounts of cardiolipin using the cardiolipin-specific fluorescence dye NAO. As shown in FIG. 14d, cardiolipin levels were reduced in 293-PLS3(F258V) cells by almost 50% compared to control or PLS3-over-expressing cells.

Targeting PLS3 reduces intracellular ATP and mitochondrial respiration.

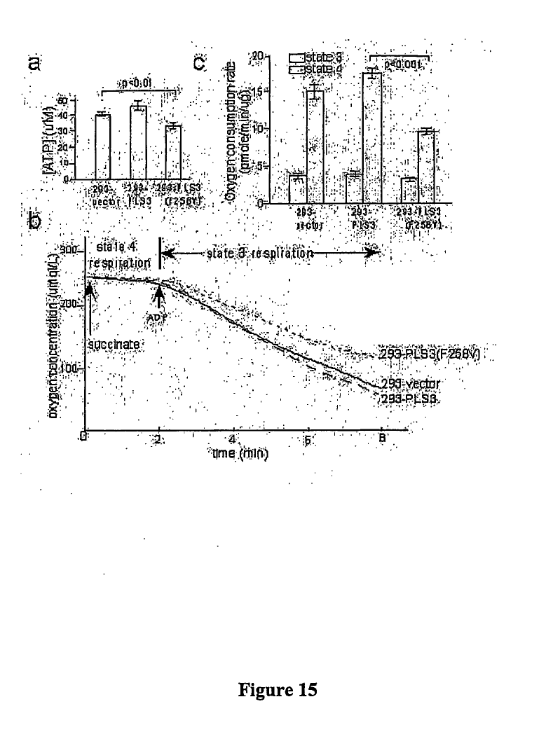

The slower growth and reduced oxidative state of 293-PLS3(F258V) cells suggests that PLS3 targeting interferes with mitochondrial respiration. The total intracellular ATP levels were measured by preparing trichloroacetic acid (TCA)-treated cell lysates for a luciferase assay (Lundin, 2000). While the ATP concentration was 15% higher in 293-PLS3 cells compared to 293-vector cells, it was 10% lower (p<0.01) in 293-PLS3(F258V) cells (FIG. 15a).

Next, oxygen consumption was measured in isolated mitochondria upon incubation with succinate (substrate for state 4 respiration) and subsequently after addition of ADP (for state 3 respiration). Compared to control and 293-PLS3 cells, the rate of oxygen consumption was reduced in 293-PLS3(F258V) cells (FIG. 15b). The rate of state 4 respiration was slightly lower in 293-PLS3(F258V) cells (3.5±0.14 pmol/min/μg) compared to 293-vector (4.05±0.21 pmol/min/μg) and 293-PLS3 cells (4.15±0.21 pmol/min/μg) (FIG. 15c). The rate of state 3 respiration in HEK293-PLS3 cells (17.5±0.71 pmol/min/μg) was higher than control cells (15±1.41 pmol/min/μg), while that of 293-PLS3(F258V) cells decreased by nearly 40% (9.85±0.21 pmol/min/μg) (FIG. 15c). Thus mitochondrial respiration was dramatically suppressed by expression of the PLS3(F258V) mutant and slightly enhanced by over-expression of wild-type PLS3.

Gross Alterations in Mitochondrial Morphology in 293-PLS3(F258V) Cells

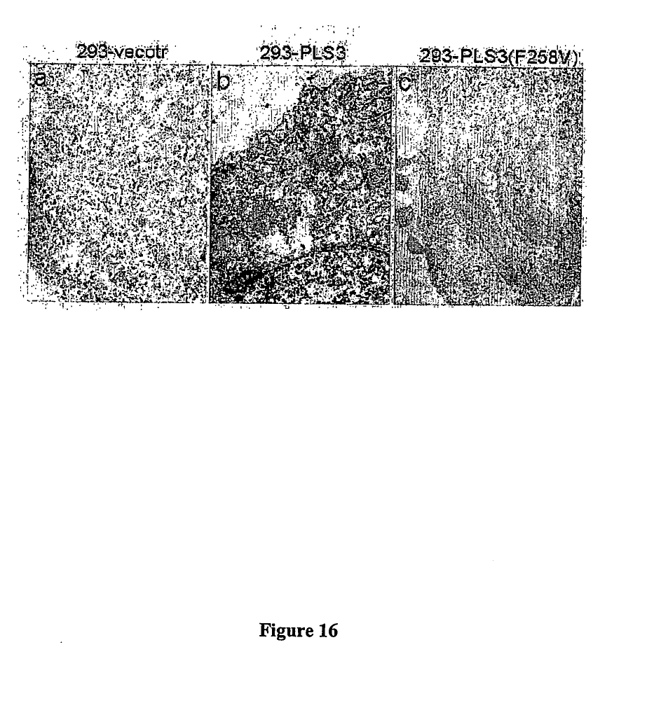

The mitochondria were next examined in these cells by electron microscopy. Mitochondria in 293-PLS3 cells were distinct from those in 293-vector cells, with fewer cristae present (FIG. 16a,b). By contrast, 293-PLS3(F258V) cells displayed few mitochondria and these were notably large in size and abnormal in shape (FIG. 16c). They contained numerous cristae tightly packed together (FIG. 16c). Thus perturbation of PLS3, either by over-expression or expression of the F258V mutant, causes abnormal mitochondrial morphology.

PLS3 Targeting Suppresses UV-Induced Apoptosis

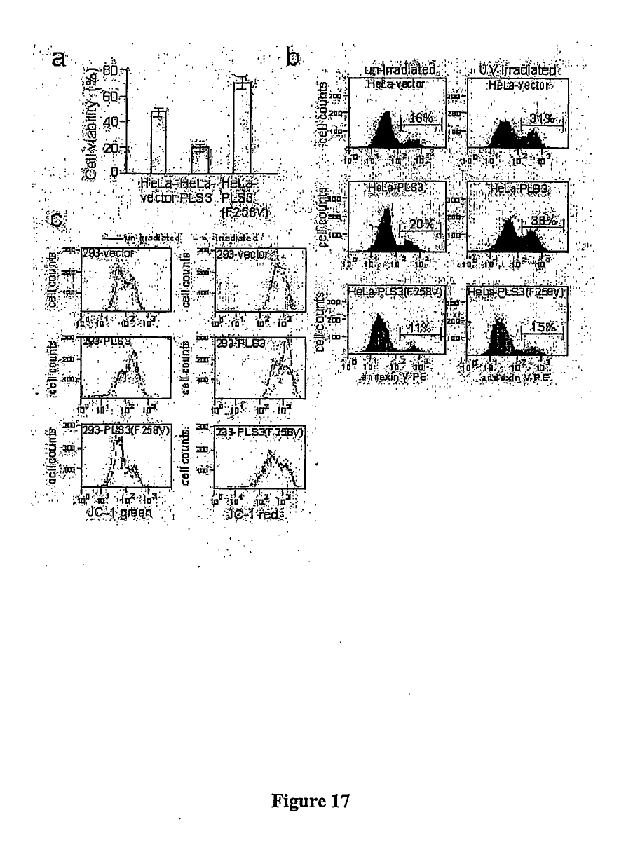

Given the central role of mitochondria in apoptosis (Brenner and Kroemer, 2000), it was determined how targeting PLS3 might affect susceptibility to apoptosis. HeLa cell transfectants Hela-vector, Hela-PLS3 and Hela-PLS3(F258V) were UV-irradiated and then assessed after 4 hours for viability using MTT assay. Cell viability after UV irradiation was 50% for HeLa-vector cells, 19.5% for HeLa-PLS3 cells, and 74% in HeLa-PLS3(F258V) cells (FIG. 17a). To confirm that the cell death was indeed apoptotic in nature, cells were also examined by Annexin V staining. As shown in FIG. 17b, UV irradiation increased the percentage of Annexin V-positive cells from 16% to 31% in HeLa-vector cells and from 20% to 38% in HeLa-PLS3 cells. By contrast, minimal change (11% to 15%) was detected in HeLa-PLS3(F258V) cells after UV irradiation (FIG. 17b). Thus over-expression of PLS3 enhanced UV-induced apoptosis, while targeting PLS3 was associated with resistance to apoptosis.

Next, the effects of this UV treatment on mitochondrial mass and potential were determined. As shown above (FIG. 14a), unirradiated 293-vector cells exhibit two populations based on JC-1 green fluorescence that reflect differences in mitochondrial mass. Exposure to UV did not dramatically change the distribution of these two population (FIG. 17c). However, the JC-1 red analysis of the mitochondrial potential exhibits a shift of the curve to left after UV irradiation. In 293-PLS3 cells, on the other hand, the JC-1 red curve shifts to right after UV (FIG. 17c). In 293-PLS3(F258V) cells, this pattern did not change after UV treatment (FIG. 17c), consistent with the resistance of these cells to UV-induced apoptosis.

UV irradiation and PLS3 over-expression increase cardiolipin in mitochondrial OM.

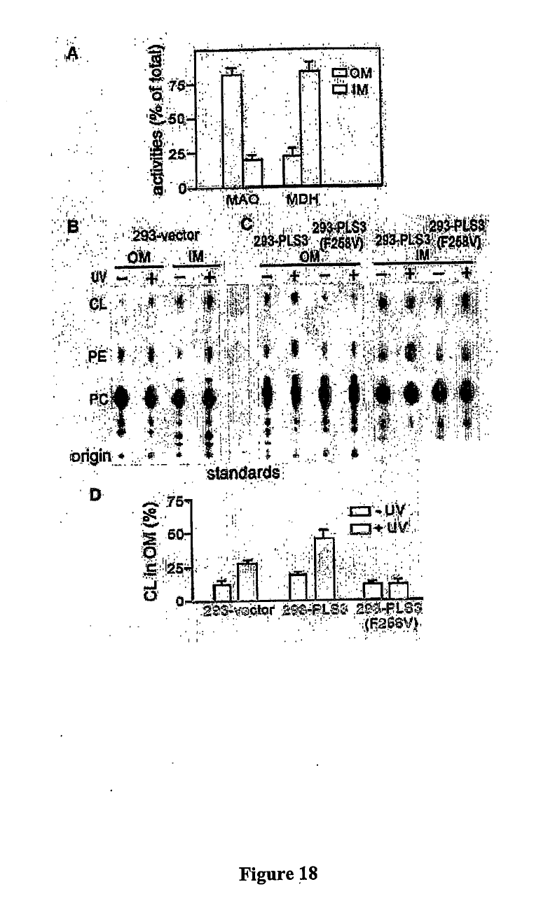

To investigate a potential mechanistic role of PLS3 in apoptosis, mitochondrial phospholipid changes were examined in cells exposed to UV radiation. Cellular phospholipids were labeled with 32P-orthophosphate, and phospholipids were analyzed by thin layer chromatography from mitochondrial membranes. The efficacy of separation of mitochondrial IM and OM was assessed by the marker enzymes MAO and MDH (Daum et al., 1982; Ragan, 1995). Both IM and OM fractions contained about 80% of the respective marker enzymes (FIG. 18a), similar to what has been reported in the literature (Ragan, 1995). The most abundant phospholipids, phosphatidylcholine (PC) and phosphatidylethanolamine (PE), did not change after UV radiation (FIGS. 18b,c) and the percentages of cardiolipin were normalized to the levels of PE. In 293-vector cells, 12.2% of the total cardiolipin (CL) was present in the OM, and UV treatment increased this percentage to 27.8% (FIGS. 18b,d). In 293-PLS3 cells, the percentage of CL in the OM was elevated at 22% and further increased following UV radiation to 45% (FIGS. 18c,d), suggesting that PLS3 facilitates CL transport from IM to OM at steady state and during apoptosis. By contrast in 293-PLS3(F258V) cells, there was minimal cardiolipin in the OM that did not increase after UV treatment (FIGS. 18c,d), suggesting that targeting PLS3 prevents mitochondrial CL transport.

PLS3 Regulates tBid-Induced Mitochondrial Cytochrome c Release

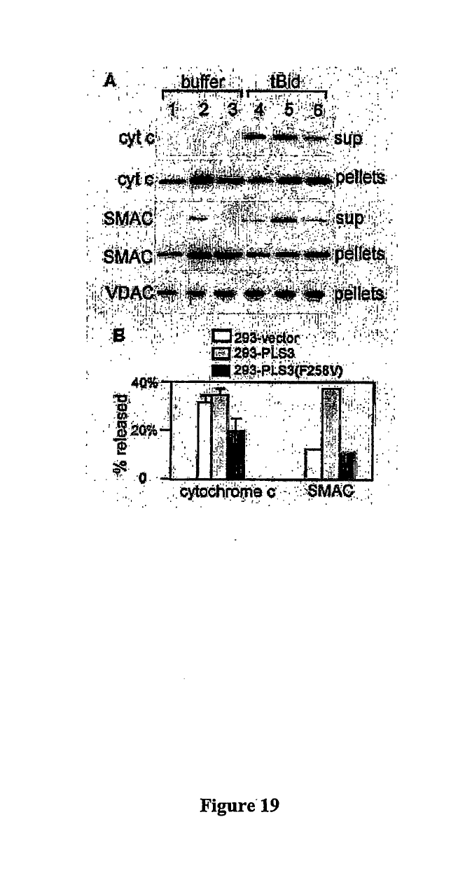

Given that CL is the mitochondrial target of tBid (Lutter et al., 2000), it was hypothesized that PLS3-mediated CL transport to the mitochondrial OM may regulate susceptibility to t-Bid-induced apoptosis. Mitochondria isolated from 293-vector, 293-PLS3, and 293-PLS3(F258V) cells were incubated with recombinant t-Bid, and the released cytochrome c were analyzed by Western blotting. Mitochondria from 293-PLS3 cells release more cytochrome c, or more sensitive to tBid-induced apoptosis than control mitochondria. By contrast, mitochondria from 293-PLS3(F258V) cells release less cytochrome c, or less sensitive to tBid than control. The loading controls by the remaining cytochrome c in the mitochondrial pellets are roughly equal (FIG. 19a). Using densitometry, the percentages of cytochrome c release were calculated to be 32.9% in 293-vector, 34.6% in 293-PLS3 and 18.9% in 293-PLS3(F258V), respectively (FIG. 19b). The amount of SMAC that is released after tBid incubation was also checked (FIG. 19c). There is also more SMAC released from 293-PLS3 mitochondria and less SMAC released from 293-PLS3(F258V) cells, confirming that changing CL in mitochondrial OM affects the sensitivity to tBid-induced apoptosis.

Discussion

The functional characterization of PLS3 in mitochondria using overexpression of wild-type or mutant PLS3 is disclosed herein. The mutant PLS3 was constructed by mutation of a conserved calcium-binding motif similarly to an inactive mutant established in PLS1 (Zhou et al., 1998). Mutant PLS3 is localized in mitochondria similar to wild-type PLS3. Cells expressing mutant PLS3 have profound morphologic and functional changes in their mitochondria. Morphologically, their mitochondria are fewer but larger, and have densely packed cristae, which is in contrast to the loose cristae in mitochondria expressing wild-type PLS3. Functionally, mitochondria expressing the PLS3(F258V) mutant have defective oxidative respiration and poor oxygen consumption. These findings are very helpful to understand the function of PLS3 in mitochondria.

This kind of abnormal mitochondria in 293-PLS3(F258V) cells has not been reported to our knowledge. The tightly packed cristae (FIG. 16c) suggest that the OM may be defective in expansion along with an increased IM expansion, thereby forcing the IM packed into such a pattern. This is in contrast to cells expressing wild-type PLS3, which displayed very loose cristae in their mitochondria compared to control (FIG. 16b), exactly the opposite of mitochondria with mutant PLS3. Based on morphologic features and the evidence that PLS3 transfers CL (and possibly other phospholipids untested in this study) from the IN to OM, it is suspected that mitochondrial membrane expansion is initiated from the IM and transported to the OM by PLS3. This theory is supported by IM localization of CL synthase (Schlame et al., 2000). When the phospholipid translocation between the IM and OM is interfered by PLS3(F258V), the mitochondrial OM can not expand properly, which results in the observed morphology. Therefore, the function of PLS3 is essential to mitochondria.

The functional defect of mitochondria expressing mutant PLS3 is apparently related with their lower amounts of CL and cytochrome c. The amount of both cytochrome c and CL in cells expressing PLS3(F258V) mutant decreased by nearly 50%, but VDAC, which is unrelated to oxidative phosphorylation, did not change. The consequence of low CL to respiration has been shown in a yeast mutant lacking CL. Moderate deficiency in mitochondrial oxidative phosphorylation was noted in yeast with no CL synthase growing at 25° C. When the temperature was shifted to 40° C., respiration was completely uncoupled from oxidative phosphorylation (Koshkin and Greenberg, 2000). The loss of cytochrome c in cells expressing PLS3(F258V) mutant further enhances the defects in oxidative phosphorylation.

The two populations of cells in the flow cytometry study with JC-1 dye suggest that the activity of PLS3 is related with mitochondrial mass and potential. Normal cells may have variations in PLS3 activities. Higher PLS3 activity shifts the mitochondrial mass to a higher level (FIG. 14a), and increases mitochondrial potential after UV irradiation, which is different from losing the potential in control 293-vector cells (FIG. 17c). Suppression of activity of PLS3 shifts the mitochondrial mass to a lower level (FIG. 14a). The population with the larger mitochondrial mass appears to be more metabolic active in oxidative phosphorylation and ATP production, as suggested by higher ATP and state 3 respiration in HEK293-PLS3 cells. The population with the lower mitochondrial mass appeared less active with lower ATP and oxygen consumption. Therefore, there is a direct correlation between the activity of PLS3 and mitochondrial respiration, and between the activity of PLS3 and sensitivities to apoptosis.

Since the levels of PLS3 affect the sensitivity to apoptosis, it is very interesting to understand how PLS3 and CL distribution affect apoptosis. Using NAO staining of CL, Garcia and Fernandez showed that cardiolipin redistribution is an early event of apoptosis (Garcia Fernandez et al., 2002). 32P-labeling of phospholipids and biochemical fractionation of mitochondrial IM and OM were used to confirm that PLS3 translocates CL from mitochondrial IN to OM (FIG. 18), which is enhanced during UV-induced apoptosis and PLS3 overexpression, and suppressed by expression of mutant PLS3.

Cardiolipin translocation to mitochondrial OM is significant for several reasons. First, cardiolipin is the mitochondrial target for tBid recruitment during apoptosis (Lutter et al., 2000). Second, cardiolipin activates the membrane permeabilization effect of Bax (Kuwana et al., 2002). Due to the localization of CL synthase (Hoch, 1992; Schlame et al., 2000), CL is mainly (estimated 90%) present in the mitochondrial IM. Thus the only two ways for tBid or Bax to get in contact with CL are that both proteins penetrate the OM, or that they interact with CL that is translocated of to the OM. Our result that UV irradiation and PLS3 overexpression increase CL in the OM supports the latter. The increased amount of CL in the OM facilitates tBid recruitment and Bax activation, which was confirmed by the enhanced tBid-induced apoptosis in 293-PLS3 cells (FIG. 19a). The degree of enhancement, however, is small due to the fact that tBid is already very powerful in inducing apoptosis. Mitochondria from 293-PLS3(F258V) cells release less cytochrome c and SMAC than those from 293-vector control in the same condition, supporting that they are more resistant to apoptosis as predicted. Hence, the regulation of PLS3-dependent CL translocation between the mitochondrial IM and OM plays a role in tBid-related apoptosis.

Materials and Methods

Wild-Type PLS3 and PLS3(F258V) Transfectants

HEK293 cells and HeLa cells were maintained in DMEM supplemented with 10% fetal calf serum and penicillin/streptomycin. Mutation of Phe258 in PLS3 to valine was carried out by site-directed mutagenesis according to manufacturer's protocol (Clontech, Palo Alto, Calif.). The cDNAs encoding wild-type PLS3 and PLS3(F258V) were cloned into the expression vector pcDNA (Invitrogen, Carlsbad, Calif.), and transfected into HEK293 or HeLa cells using the calcium phosphate precipitation method. Transfected cells were selected with G418 (1 mg/ml) and resistant clones were picked and expanded for Western analysis using anti-PLS3 antibody. Polyclonal antibody was raised in rabbits against the N-terminal 50 amino acids of PLS3 (Zymed laboratory, South San Francisco, Calif.) and purified by affinity chromatography before use.

Flow Cytometry Analysis

Flow cytometry was performed on a FACScan using Cell Quest software (Becton Dickinson, San Jose, Calif.) at the University of Utah core facility. To determine mitochondrial mass and transmembrane potential, cells were incubated with 10 μg/ml JC-1 or 200 nM Rosamine (Molecular Probes, Eugene, Oreg.) followed by flow cytometry according to the manufacturer's instructions. Cell cycle analysis was performed on ethanol-fixed cells stained with propidium iodide (50 μg/ml) followed by flow cytometry analysis.

Subcellular Fractionation and Preparation of Mitochondria and Mitoplasts

Mitochondria were isolated by differential centrifugation. Briefly, mouse liver was incubated in buffer containing 300 mM sucrose, 10 mM Tris-HCl (pH 7.5), 5 mM EDTA, 0.1% BSA and 1 mM PMSF for 5 min on ice. Cells were disrupted in a homogenizer by douncing 10 strokes, and centrifuged 1000×g for 5 min, 10,000×g for 10 min, and 30,000×g for 60 min, for collection of intact cells/nuclei, crude mitochondria and microsomes, respectively. The final supernatant represented the cytoplasmic fraction.

To further purify mitochondria for outer and inner membrane isolation, crude mitochondria were loaded on a sucrose gradient of 1-2 M in the mitochondrial isolation buffer, and centrifuged at 100,000×g for 90 min in a table-top ultracentrifuge (Beckman OptimaTMMax). Mitoplasts were prepared by osmotic disruption of the mitochondrial outer membrane (OM) with 10 mM HEPES/KOH (pH 7.4), and centrifuged at 20,000×g for 15 min. To determine the purity of separation, the activities of monoamine oxidase (MAO) and malate dehydrogenase (MDH) were measured, as OM and inner membrane (IM) markers (Ragan, 1995).

Cardiolipin and Phospholipid Analysis

For cardiolipin quantitation, cells were fixed with 4% formaldehyde in PBS for 10 min. Cardiolipin was stained with 30 uM [10-N-nonyl-3,6-bis (dimethylamino) acridine orange] (NAO, Molecular Probes) and the fluorescence intensities at 590 nm were measured using a Bio-Tek microplate reader. In pilot studies, it was found that an NAO concentration of 15 μM was sufficient to saturate mitochondrial cardiolipin in 105 cells. For phospholipid analysis, cells were fixed with 1% formaldehyde for 15 min at room temperature and washed twice with cold PBS. Phospholipids were extracted according to the standard method of Bligh and Dyer (Bligh, 1959). Lipids were analyzed by TLC in a solvent system of chloroform-methanol-water-ammonium hydroxide 120:75:6:2 (v/v) (Fine and Sprecher, 1982) using Whatman silica TLC plates (Fisher Scientific, Pittsburgh, Pa.).

Cell Viability and Apoptosis

Cells were irradiated with unfiltered UV-B lamps (National Biological Corp., Twinsburg, Ohio) at 4 J/m²/sec over 2 min. Cell viability assays using 3-(4,5-dimethylthiazol-2-yl)-2,5-diphenyl tetrazolium bromide (MTT, Chemicon International Corp. Temecula, Calif.) were performed as described (Mosmann, 1983). Staining with Annexin V-PE (BD Pharmingen, San Diego, Calif.) and flow cytometric analysis was performed according to the manufacturer's recommendations.

Intracellular ATP and Oxygen Consumption

For ATP quantitation, cells (1×107) were washed with cold PBS, lysed with 5% TCA, and the resulting supernatants were analyzed using a commercial ATP kit (Biotherma, Sweden) and a MLX Microtiter plate Luminometer (Dynex Technologies, Inc). State 3 and 4 mitochondrial oxygen consumption was measured at 250 C using a Mitocell connected to a two-channel dissolved oxygen measuring system (model #782, Strathkelvin Instrument, Glasgow, UK). Mitochondria were isolated from cells as described (Gottlieb et al., 2002). Briefly, cells were washed once with mitochondria isolation buffer (MIB) containing 200 mM mannitol, 70 mM sucrose, 1 mM EGTA, and 10 mM Hepes (pH 7.4). After incubating 10 min in ice-cold MIB containing 0.5 mg/ml BSA, cells were then homogenized using a syringe-driven 25 G needle. The homogenate was spun at 800×g for 10 min at 40 C. The supernatant was collected and spun at 10,000×g for 10 min at 40 C, and the pellet was resuspended in MIB containing BSA. Mitochondrial fractions (50 μg protein) were then diluted in respiration buffer (225 mM manitol, 70 mM sucrose, 10 mM KH₂PO₄ and 1 mM EGTA, pH 7.2). For state 4 (steady state) respiration, succinate (Sigma) was injected into the chamber at a final concentration of 7 mM and oxygen concentrations were monitored for 2 min. To measure state 3 (ADP-stimulated) respiration, ADP (Sigma) was then added at a final concentration of 150 μM for another 6 min.

Electron Microscopy

Cells were fixed overnight at 4° C. in 0.1 M sodium cacodylate with 2.5% glutaraldehyde and 1% paraformaldehyde, with the addition of 2.4% sucrose and 8 mM calcium chloride, then washed twice with 0.1 M sodium cacodylate and re-fixed for 45 min in 2% osmium tetroxide in 0.1 M cacodylate buffer. After washing in water, cells were stained enbloc with saturated aqueous uranyl acetate for 1 hour. After dehydration in a graded series of ethanol, infiltration was carried out through a series of ethanol: Spurr's plastic ratios and finally embedded in straight Spurr's plastic. Blocks were polymerized overnight in a 60° C. oven, and sectioned with a diamond knife to a thickness of 60-80 nm, and sections picked up on 135 hex mesh grids. Sections were stained in saturated aqueous uranyl acetate followed by Reynold's lead citrate. All electron micrographs were taken on a Hitachi H-7100 transmission electron microscope at 75 KV.

IV. Phospholipid Scramblase 3 is the Mitochondrial Target of PCK-δ Induced Apoptosis

Materials and Methods:

HEK293 and HeLa cells were grown in DMEM supplemented with 10% fetal bovine serum. The cDNAs of PLS3 and PLS3(F258V) were cloned into pcDNA3.1 vector (Invitrogen, Carlsbad, Calif.) for expression in mammalian cells. PKC-δ was cloned into pCMV/Mito/myc vector (Invitrogen, Carlsbad, Calif.) to construct the mitochondrial targeted PKC-δ. Go6976, recombinant PKC-δ enzyme and c-abl antibody were purchased from Calbiochem (San Diego, Calif.). Antibodies against phosphoserine (PS), phosphothreonine PT), phosphotyrosine (PY), PKC-δ and the PKC-δ inhibitor, rottlerin, were purchased from Sigma (St. Louis, Mo.).

In Vitro Phosphorylation and Kinetics Assay.

PLS3 protein was overexpressed as His-tagged protein by pQE (Qiagen) vector, and then purified with nickel-column in 8M urea. The PLS3 protein was eluted in urea and dialyzed against 2M urea before usage. Immediately before in vitro phosphorylation, PLS3 was further diluted to less than 0.2 M urea in the final phosphorylation reaction. In vitro phosphorylation was performed with 0.1 μg PKC-δ, γ-[32P]-ATP and 1 μg of recombinant PLS3 protein in a reaction buffer as described in the manufacturer's protocol (Calbiochem, San Diego, Calif.). The reaction mix was incubated in room temperature and stopped at various time points, or after 20 min in the kinetics study, with SDS loading buffer. The phosphorylated products were separated by SDS-PAGE and analyzed by autoradiography.

Immunoprecipitation.

Immunoprecipitation was performed with affinity purified PLS3 antibody at 1:100 dilution followed by Western analysis with PS, PT, PY and PKC-δ antibodies. The Western blot was developed with chemiluminescence (Pierce, Rockford, Ill.).

Apoptotic Assays.

Apoptotic assays were performed with annexinV-PE (BD-PharMingen) per manufacturer's protocol. The TUNEL assay (terminal deoxynucleotidyltransferase-mediated dUTP-biotin nick end labeling) was performed according to the manufacturer's protocol (Roche, Switzerland).

Subcellular Fractionation.

Subcellular fractionation was performed by differential centrifugation. In brief, 107 cells were incubated in buffer containing 300 mM sucrose, 10 mM Tris (pH 7.5), 5 mM EDTA, 0.1% BSA and 1 mM PMSF protease inhibitor for 5 min on ice. Cells were disrupted by passage through a 25 G needle or dounced for 10 strokes, and then were centrifuged at 1000×g for 5 minutes, 10,000×g for 10 minutes, and 30,000×g for 60 minutes, for collection of intact cells/nuclei, crude mitochondria and microsomes, respectively. The final supernatants were used as cytosols. The mitochondria and the cytosolic fractions were analyzed for cytochrome c release with Western blot.

Immunofluorescent Staining.

Cells were fixed with 4% paraformaldehyde in phosphate-buffered saline (PBS) for 5 min. Cells were washed and permeabilized with 0.2% Triton X-100 in PBS for 2 min. Non-specific reaction was blocked with 3% BSA for 15 min. Primary antibody against PKC-δ was used at 1:100 and secondary antibody with FITC was used at 1:200 dilution. The stained cells were visualized by Nikon fluorescence microscope Eclipse TE300.

Results

PLS3 is a phosphoprotein and the phosphorylation increases after UV irradiation.

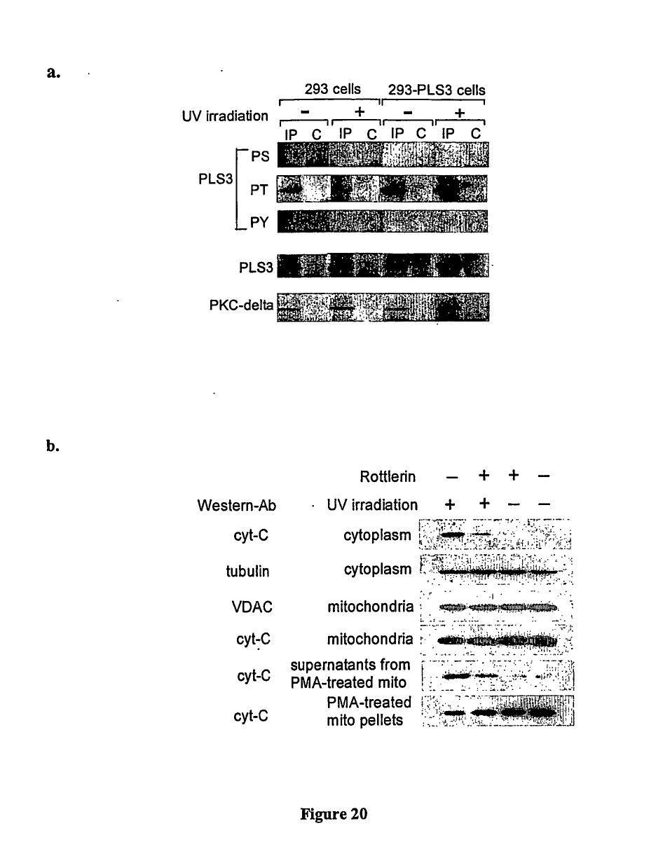

In order to investigate the regulation of PLS3 activity, the phosphorylation status of PLS3 during UV-induced apoptosis were studied. Immunoprecipitation (IP) in HEK293 and HEK293-PLS3 cellular extracts were performed with an antibody to PLS3, and probed the Western blot with antibodies specific for phosphoserine (PS), phosphothreonine (PT) and phosphotyrosine (PY). The immunoprecipitated PLS3 was recognized by the anti-PT antibody, but not by anti-PS or anti-PY (FIG. 20a), indicating that PLS3 is phosphorylated at the threonine residue. UV irradiation increased the PT signals in both the control HEK293 and HEK293-PLS3 cells. The same blot was probed with PLS3 antibody as a loading control, and revealed minimal difference in the amount of PLS3 protein before and after UV irradiation (FIG. 20a).

PKC-δ Physically Interacts with PLS3.

The kinase that phosphorylates PLS3 was then sought. Based on the observation that PLS1 is phosphorylated by PKC-8, we studied whether PLS3 is the mitochondrial substrate of PKC-δ. It was first reconfirmed that PKC-δ is essential for UV-induced apoptosis by blocking PKC-δ with a PKC-δ inhibitor, rottlerin, in our system. The HEK293 cells were fractionated and the isolated mitochondria and cytosols were analyzed for cytochrome c release by Western blot. This study confirmed that cytochrome c was released to cytosol after UV irradiation, and this release was inhibited by rottlerin (FIG. 20b). The loading controls of the mitochondria or cytoplasms showed equal amounts of voltage-dependent anion channel (VDAC) or tubulin. To examine the effect of translocated PKC-δ to mitochondrial cytochrome c release, mitochondria were washed with mitochondrial isolation buffer to remove rottlerin. The washed mitochondria were then treated with phorbol ester PMA to achieve maximal activation of PKC-δ, and the released cytochrome c after PMA treatment was studied. The mitochondria from non UV-irradiated cells had very minimal cytochrome c release, indicating very little PKC-δ present. The mitochondria from UV-irradiated but not rottlerin-treated cells released the most cytochrome c after PMA treatment, confirming the existence of PKC-δ in mitochondria after UV irradiation (FIG. 20b bottom two panels). The mitochondria from UV and rottlerin-treated cells released more cytochrome c than those from un-irradiated cells, but less than those from UV irradiated cells, indicating that the amount of PKC-δ translocated to mitochondria decreased with rottlerin treatment. Therefore, there was a direct correlation between the amount of translocated PKC-δ and the released cytochrome c.

It was then studied whether PLS3 interacted with PKC-δ. PLS3 was immunoprecipitated from the cell lysates of UV-irradiated and non-irradiated HEK293 cells. The precipitates were analyzed by immunoblotting for PLS3 and for PKC-δ. PCK-δ was observed in the blot, indicating that PLS3 and PKC-δ co-immunoprecipitate. Further, this co-immunoprecipitation increased in cells following UV irradiation (FIG. 20a, bottom panel). However, the amount of the PKC-δ that co-immunoprecipitated with PLS3 is only about 5-10% of total cellular PKC-δ based on the estimation from a PKC-δ blot for comparison (not shown). This was not surprising since only a portion of cellular PKC-δ translocates to mitochondria during apoptosis.

Because PKC-δ also interacts with c-abl kinase and phosphorylates c-ab1, the same IP blot was probed with c-abl antibody to determine whether PKC-δ, PLS3, and c-abl formed a complex. However, no c-abl signal was detected in the immunoprecipitates of PLS3 (not shown). The fact that PLS3 was not phosphorylated at the tyrosine residue also argues against PLS3 as a substrate of c-abl kinase.

PKC-δ Phosphorylates PLS3 In Vitro.

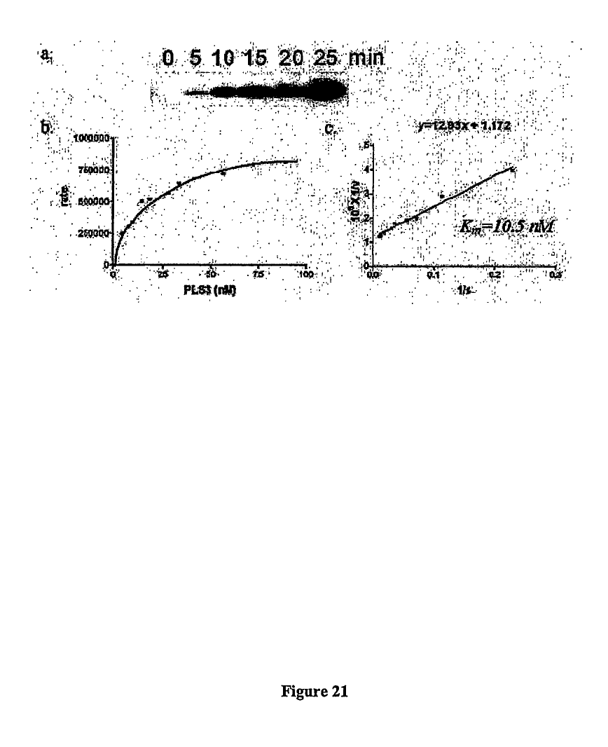

Next PLS3 was evaluated as a substrate for PKC-δ using recombinant proteins. The PLS3 protein was a high-affinity substrate for PKC-δ and the phosphorylation steadily increased over the first 25 minutes (FIG. 21a). Various concentrations of PLS3 were tested, and it was determined that the km is 10.5 nM (FIGS. 21b,c). This is a very high affinity for PKC-δ toward PLS3 compared with other physiologic substrates of the PKC family.

Phorbol ester PMA induces apoptosis in cells overexpressing wild-type PLS3.

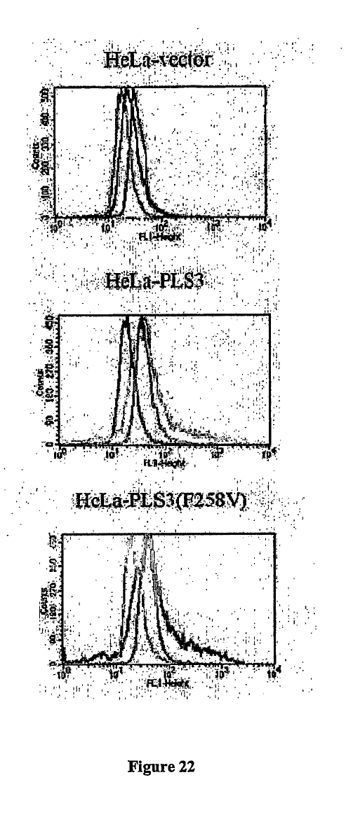

If PLS3 is a direct downstream effector of PKC-δ in the PKC-δ-induced apoptotic pathway, then overexpression of PLS3 might enhance apoptosis induced by PKC-δ activation. HeLa-PLS3 were incubated with phorbol ester PMA, and analyzed for apoptosis with TUNEL assay and flow cytometry. PMA treatment of the control HeLa cells did not induce any apoptosis, but treatment of the HeLa-PLS3 cells shifted the curve to right (FIG. 22). To eliminate the effect of classic PKCs (a, 3, and y) that are also stimulated by PMA, they were treated with a combination of PMA and the indolocarbazole Go6976, which is a potent inhibitor for PKC-α, β, and γ, but does not affects PKC-δ. The curve of HeLa-PLS3 cells had a similar shift to right like that of PMA alone, indicating that this apoptotic effect by PMA is not related with the classic PKCs. These data support that the overexpression of the PKC-δ substrate PLS3 makes the mitochondrial effect of PKC-δ so dominant to induce apoptosis.

A mutant of PLS3 was also constructed by mutating Phe258 to Val. This Phe258 is in a highly conserved calcium-binding motif, and mutation of the corresponding Phe at PLS1 completely abolishes the activity of PLS1. Overexpressing PLS3(F258V) mutant in HeLa cells resulted in a higher baseline apoptosis probably due to interfering with mitochondrial functions. PMA treatment of HeLa-PLS3(F258V) cells decreased apoptosis by shifting the curve to left (FIG. 22, bottom panel). When the same cells were treated with both PMA and Go6976, the left shift by PMA was inhibited, indicating that the left shift was due to activation of a survival signal from a classic PKC-related pathway.

Mitochondrial targeted PKC-δ Induced Apoptosis in HeLa-PLS3 Cells.

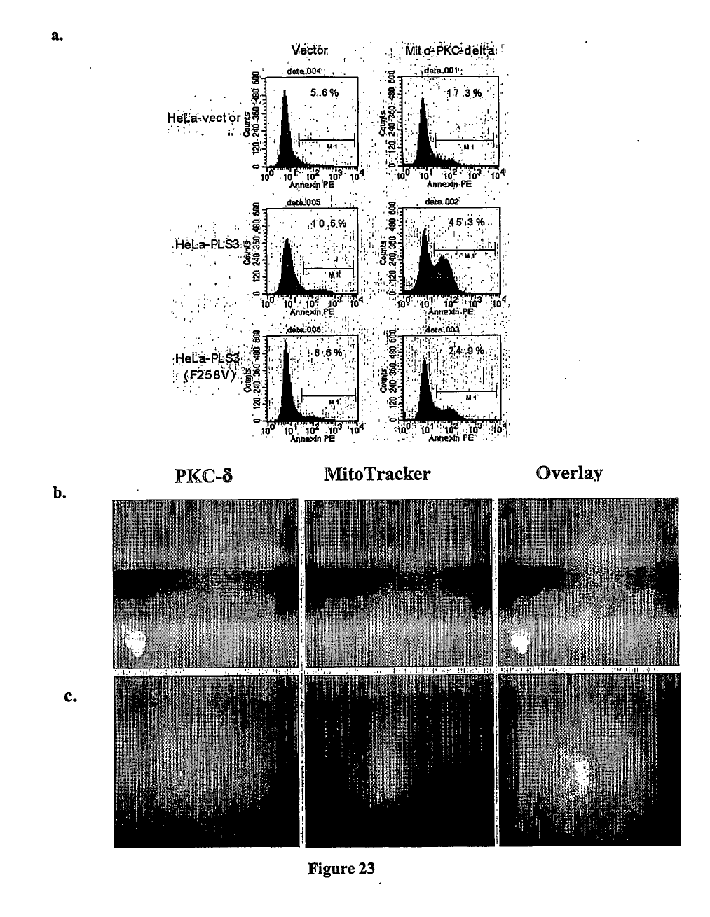

Because PMA activates many other isoforms of PKCs and does not represent PKC-δ-specific stimulation, a mitochondrial targeted PKC-δ was constructed by inserting the mitochondrial targeting sequence of cytochrome oxidase at the N-terminus of PKC-δ. The construct was transfected into HeLa cells and G418-resistant clones were selected. No clones were obtained after 10 days of selection; while a control construct replacing PKC-δ with EGFP resulted in many clones with EGFP expressed in the mitochondria (not shown). This indicates that overexpression of PKC-δ in the mitochondria is toxic to cells. The mitochondrial targeted PKC-δ was expressed into HeLa-control, HeLa-PLS3 and HeLa-PLS3(F258V) cells, and studied apoptosis with annexinV-PE 36 hours after transfection. Expression of the mitochondrial targeted PKC-δ induced 17.3% of cell death in the control HeLa cells, 45.3% in HeLa-PLS3 cells, indicating that PLS3 enhanced the apoptotic effect of PKC-δ in the mitochondria. HeLa cells expressing PLS3(F258V) had only 24.9% of apoptosis in a similar study, far less effective in enhancing apoptosis than HeLa-PLS3 cells (FIG. 23a).

To prove that the PKC-δ was expressed in the mitochondria, HeLa cells were stained 3 days after transfection with PKC-δ antibody and co-localized with MitoTracker Red dye. In the vector-transfected cells, PKC-δ was diffusely present in the cytosol and perimembranous area, and did not overlay with MitoTracker Red (FIG. 23b). MitoTracker Red staining of the Mito-PKC-δ-transfected cells revealed all mitochondria clustered around the perinuclear area, a pattern reminiscent of early apoptosis. PKC-δ staining of the Mito-PKC-δ-transfected cells displayed more PKC-δ in the perinuclear area which were overlaid with the MitoTracker Red dye. This represents the overexpressed mitochondrial targeted PKC-8. There was also a less abundant cytosolic signal, likely from the endogenous PKC-δ (FIG. 23c).

Discussion:

PLS3 is the substrate of PKC-δ in the mitochondria. Overexpression of PLS3 sensitizes cells to apoptotic stimuli, presumably through augmentation of the effect of PKC-δ. This is best supported by the induction of apoptosis in HeLa-PLS3 cells with PMA. In the normal situation, PMA activates many different PKCs, which lead to activation of both cell survival and death signals. Depending on which pathway prevails, cells will become live or dead. When PLS3 is overexpressed, the PKC-δ-related PLS3 activation becomes dominant to induce apoptosis. This effect was not affected by a potent classic PKC inhibitor Go6976, confirming that it is indeed related with PKC-δ. The inactive PLS3(F258V) mutant failed to enhance the apoptotic effect of PKC-δ, and PMA actually protected HeLa-PLS3(F258V) cells through losing the downstream effector of apoptotic PKC-δ and activation of survival signals induced by PMA. This was confirmed by the addition of Go6976, which inhibited the survival effect of PMA.

It is impossible to state that PLS3 is the only substrate of PKC-δ in the mitochondria. If so, blocking PLS3 with the dominant negative mutant PLS3(F258V) would completely prevent apoptosis induced by the mitochondria targeted PKC-8, which appeared not the case. However, the fact that the cells expressing PLS3(F258V) had a high baseline cell death makes the interpretation difficult, because they may die from the overexpressed PKC-δ in the mitochondria, or they die from poor mitochondrial functions. It is impossible to rule out the possibility that PKC-δ phosphorylates other substrate in mitochondria to induce apoptosis. Another question is whether PKC-δ is the only kinase that phosphorylates PLS3. The answer is likely negative as well. The fact that PLS3 has a baseline phosphorylation even before the induction of apoptosis (FIG. 20a) indicates that PLS3 may be phosphorylated by other kinases, but the UV-enhanced phosphorylation is likely associated with further increased PLS3 activity. With the important role of PLS3 in mitochondria apoptosis, understanding the mechanism of PLS3 regulation is very important. PKC-δ is the kinase that contributes to the activation of PLS3.

It is interesting that both PLS1 and PLS3 are phosphorylated by PKC-δ during apoptosis but at different locations. Although the significance of PLS1 phosphorylation is unclear, it has been proposed that PLS1 might be related with the translocation of PS to the outer leaflet of plasma membrane. PLS3, present in the mitochondria, translocates cardiolipin to the outer membrane of the mitochondria, an interesting analogy when you consider that mitochondria are evolved from a prokaryote trapped inside of an eukaryote cell in the early stage of evolution. The phospholipid scramblase family is highly conserved in almost all eulcaryotes, including yeast, Drosophila and C. elegans. Genetic studies in those lower organisms will be very helpful to dissect the mechanism of PLS regulation.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A method of increasing the resistance of a cell to apoptosis comprising the step of:
inhibiting expression or activity of PLS3 in the cell.

2. The method of claim 1, wherein inhibiting expression or activity of PLS3 in the cell includes introducing a non-functional PLS3 into the cell.

3. The method of claim 2, wherein the sequence of the non-functional PLS-3 is developed from PLS3α (SEQ ID NO: 1) or PLS3β (SEQ ID NO: 3).

4. The method of claim 3, wherein the non-functional PLS3 includes a mutation in its calcium-binding motif.

5. The method of claim 4, wherein the non-functional PLS3 includes the sequence of SEQ ID NO: 12.

6. The method of claim 4, wherein the non-functional PLS3 includes the sequence of SEQ ID NO: 13.

7. The method of claim 2, wherein the non-functional PLS3 is introduced into the cell by transfecting the cell with a vector expressing a dominant negative non-functional PLS-3.

8. The method of claim 7, wherein the sequence of the non-functional PLS3 is developed from PLS3α (SEQ ID NO: 1) or PLS3β (SEQ ID NO: 3).

9. The method of claim 8, wherein the non-functional PLS3 includes a mutation in its calcium-binding motif.

10. The method of claim 9, wherein the non-functional PLS3 includes the sequence of SEQ ID NO: 12.

11. The method of claim 9, wherein the non-functional PLS3 includes the sequence of SEQ ID NO: 13.

12. The method of claim 1, wherein the step of: inhibiting expression or activity of PLS3 in the cell includes exposing the cell to an inhibitory oligonucleotide.

13. A method of inducing cellular apoptosis comprising the step of increasing the amount of PLS3 present in a cell.

14. The method of claim 13, wherein inducing cellular apoptosis includes introducing PLS3 into the cell.

15. The method of claim 14, wherein the sequence of the PLS-3 is either PLS3α (SEQ ID NO: 1) or PLS3β (SEQ ID NO: 3).

16. The method of claim 13, wherein the non-functional PLS-3 is introduced into the cell by transfecting the cell with a vector capable of expressing PLS-3.

17. The method of claim 16, wherein the sequence of the PLS-3 expressed is either PLS3α (SEQ ID NO: 1) or PLS3β (SEQ ID NO: 3).

18. An isolated nucleic acid comprising a sequence that encodes a polypeptide with the amino acid sequence of SEQ ID NO: 1.

19. The isolated and purified nucleic acid of claim 1, comprising the sequence of SEQ ID NO: 2 or a degenerate variant of SEQ ID NO: 2.

20. An isolated nucleic acid comprising a sequence that encodes a polypeptide having the sequence of SEQ ID NO: 1, or SEQ ID NO: 1 with conservative amino acid substitutions.

21. A method of identifying a compound that modulates the function of PLS3 in a cell, the method comprising the steps of: providing a cell expressing PLS3, contacting the cell with a test compound, and determining whether the test compound modulates the expression of PLS3, wherein the induction of apoptosis is an indication that the test compound upregulates PLS3 or interferes with its function.

22. A method of identifying a compound that modulates the function of PLS3 in a cell, the method comprising the steps of: providing a cell expressing PLS3, contacting the cell with a test compound, exposing the cell to an apoptosis-inducing stimulus, and determining whether the test compound modulates the expression of PLS3, wherein resistance to apoptosis is an indication that the test compound downregulates PLS3 or interferes with its function.


SEQUENCE LISTING (13 sequences)

SEQ ID NO: 1 - protein, 292 aa, Homo sapiens
MAGYLPPKGYAPSPPPPYPVTPGYPEPALHPGPGQAPVPAQVPAPAPGFALFPSPGPVALGSAAPFLPLPGVPSGLEFLVQIDQILIHQKAERVETFLGWETCNRYELRSGAGQPLGQAAEESNCCARLCCGARRPLRVRLADPGDREVLRLLRPLHCGCSCCPCGLQEMEVQAPPGTTIGHVLQTWHPFLPKFSIQDADRQTVLRVVGPCWTCGCGTDTNFEVKTRDESRSVGRISKQWGGLVREALTDADDFGLQFPLDLDVRVKAVLLGATFLIGPAFSWCLLRFLCLM

SEQ ID NO: 2 - DNA, 984 bp, Homo sapiens
catggcaggctacttgccccccaaaggctacgccccttcgcccccacctccctaccctgtcacccctgggtacccggagccggcgctacatcctgggcccgggcaggcgccagtgcccgcccaggtacctgccccagctcccggcttcgccctcttcccctcgcctggccccgtggccttggggtctgctgcccccttcttgccactgccaggggtgccttctggcctcgaattcctggtgcagattgatcagattttgattcaccagaaggctgagcgagtggaaacgttcctaggctgggagacctgtaatcggtatgaactgcgctctggggccgggcagcccctgggtcaggcggccgaggagagcaactgctgcgcccgtctgtgctgtggcgcccgccggccgctgcgtgtccgcctggccgaccccggggaccgtgaggtgctgcgtttgctccgcccgctgcactgtggctgcagctgctgcccctgtggcctccaggagatggaagtacaggctccaccaggcaccaccattggccacgtgctacagacctggcatcccttcctccccaagttctccatccaggatgccgatcgccagacagtcttgcgagtggtggggccctgctggacctgtggctgtggcacagacaccaactttgaggtgaagactcgggatgaatcccgcagtgtgggccgcatcagcaagcagtgggggggcctggtccgagaagccctcacagatgcagatgactttggcctacagttcccgctggacctggatgtgagggtgaaggctgtgctgctgggagccacattcctcattggccctgccttcagctggtgcttgctgcgattcctgtgccttatgtaactgcccttccttcccttgccctaggaaaaaggctgcatctttatatgttacattcatataaactttgtaactttttggaaaaaaaaaaaaaaaaaaaaaaaaaa

SEQ ID NO: 3 - protein, 295 aa, Homo sapiens
MAGYLPPKGYAPSPPPPYPVTPGYPEPALHPGPGQAPVPAQVPAPAPGFALFPSPGPVALGSAAPFLPLPGVPSGLEFLVQIDQILIHQKAERVETFLGWETCNRYELRSGAGQPLGQAAEESNCCARLCCGARRPLRVRLADPGDREVLRLLRPLHCGCSCCPCGLQEMEVQAPPGTTIGHVLQTWHPFLPKFSIQDADRQTVLRVVGPCWTCGCGTDTNFEVKTRDESRSVGRISKQWGGLVREALTDADDFGLQFPLDLDVRVKAVLLGATFLIDYMFFEKRGGAGPSAITS

SEQ ID NO: 4 - DNA, 1538 bp, Homo sapiens
catggcaggctacttgccccccaaaggctacgccccttcgcccccacctccctaccctgtcacccctgggtacccggagccggcgctacatcctgggcccgggcaggcgccagtgcccgcccaggtacctgccccagctcccggcttcgccctcttcccctcgcctggccccgtggccttggggtctgctgcccccttcttgccactgccaggggtgccttctggcctcgaattcctggtgcagattgatcagattttgattcaccagaaggctgagcgagtggaaacgttcctaggctgggagacctgtaatcggtatgaactgcgctctggggccgggcagcccctgggtcaggcggccgaggagagcaactgctgcgcccgtctgtgctgtggcgcccgccggccgctgcgtgtccgcctggccgaccccggggaccgtgaggtgctgcgtttgctccgcccgctgcactgtggctgcagctgctgcccctgtggcctccaggagatggaagtacaggctccaccaggcaccaccattggccacgtgctacagacctggcatcccttcctccccaagttctccatccaggatgccgatcgccagacagtcttgcgagtggtggggccctgctggacctgtggctgtggcacagacaccaactttgaggtgaagactcgggatgaatcccgcagtgtgggccgcatcagcaagcagtgggggggcctggtccgagaagccctcacagatgcagatgactttggcctacagttcccgctggacctggatgtgagggtgaaggctgtgctgctgggagccacattcctcattgactacatgttctttgagaagcgaggaggcgctgggccctctgccgtcaccagttagaggccaccatggtgtgaggagaccatcacctcgaccagaactccagatggtcacctgccctggcccctcctctgggcagcccctttcctccatgtacactgcaggggacagaaggggggccccatccctaccctactccctggccgcctgcccctgtggttcccaaggaggggtatgtatgagagccgctctcctgctacctcccaccactgtcccagcagtccctcggcacacaggcatatcagctttcacactttccccatgcactctctcccacccccttccagggcctctgctccaaaggaggcctctggaacccaggactctggggttttacaagagggctggggtgtggaagggcaagctgcaccaaagacggtggatatagccaccgcccccccgccgctgcctagcatctgcttggccaattagttcagcctcagaccatggcactttgagggggtctctacctccccatcaacagctgcagggggaccccagtgccaacttcctctcccactagggccctgccttcagctggtgcttgctgcgattcctgtgccttatgtaactgcccttccttcccttgccctaggaaaaaggctgcatctttatatgttacattcatataaactttgtaactttttgg

SEQ ID NO: 5 - protein, 296 aa, Mus
MAGYLPPKGYAPSPPPPYPVPSGYPEPVALHPGPGQAPVPTQVPAPAPGFALFPSPGPVAPGPPAPGVPLPGVPPGLEFLVQIDQILIHZKAERVETFLGWETCNMYELRSGTGQQLGQAAEESNCCARLCCGARRPFRIRLADPGDREVLRLLRPLHCFCSCCPCGLQEMEVQAPPGTTIGHVLQTWHPFLPKFSILDADRQPVLRVVGPCWTCGCGTDTNFEVKTKDESRSVGRISKQWGGLLREALTDADDFGLQFPVDLDVKVKAVLLGATFLIDYMFFEKRGGAGPSAITS

SEQ ID NO: 6 - protein, 263 aa, Drosophila
MRLNKSAKRKGYEFYMSDEDRSIIFAGDWMSIPTGIPNCPRGLEYLTTIDQLLVKQKVELLEAFTGFETNNKFTIKNALGQKVYFAAEDNDCCTRNCCGPARPFDMRVFDNFQQEVIHMHRPLACSSCLFPCCLQSIEVSAPPGNVIGTIEQEWSICSPSFRILNHVGDTVMRIEGPFCTFSLCGDVEFNVVSLTGEKIGKISKQWSGLAREIFTDADFFGINFPLDLDVRMKAVLLGATFLIQKFVLVVKMQALQVLALLLL

SEQ ID NO: 7 - protein, 265 aa, Caenorhabditis elegans
MDQLFYNGPPDYVEIEDRAITTEPGASIQAAPGAIWMEALPAIEGIPAGLEYLAYLDTVMVHQVKEVLEILTGWESKNKYAIRNKNGQQCYYAFEESNAWERQCCGSQRGFTMHIVDNFNKNVLIVKRELRCCADSFCGCLLGLQNICTIESLSTGLLGTVLQGYGCTNSFYNILDKCGNLIFLVDGQGCCTSCCCEDKDFTIKTPDSSVPIGSITKKWGGLLREAFTDADTFAVTFPIDLDVKLKAILLGATFLIDFVEFENNN

SEQ ID NO: 8 - protein, 15 aa, Homo sapiens
CDTNFEVKTRDESRS

SEQ ID NO: 9 - DNA, 20 bp, Artificial sequence
  Mitochondrion-specific PCR primer
aacatacccatggccaacct

SEQ ID NO: 10 - DNA, 20 bp, Artificial sequence
  Mitochondrion-specific PCR primer
ggcaggagtaatcagaggtg

SEQ ID NO: 11 - DNA, 618 bp, Homo sapiens
  misc_feature (519)..(519): n is a, c, g, or t
ggcgccgcccaagagaccctgggccggcgccgggcgcagctgcctctccgtctttgtgtctgtctctgtgtctgtctggctatctccgagtttgcctccgcttccagaactaagccacccagacaccatcatcccgaaaaccccagcccttctcccatggcaggctacttgccccccaaaggctacgccccttcgcccccacctccctaccctgtcacccctgggtacccggagccggcgctacatcctgggcccgggcaggcgccagtgcccgcccaggtacctgccccagctcccggcttcgccctcttcccctcgcctggccccgtggccttggggtctgctgcccccttcttgccactgccaggggtgccttctggcctcgaattcctggtgcagattgatcagattttgattcaccagaaggctgagcgagtggaaacgttcctaggctgggagacctgtaatcggtatgaactgcgctctggggccgggcagccccttggtcatgcggccgangagagcaactgctgcgcccgtctgtgctgtggcgcccgccgggcgctgcgtgtcccgctggccgaaccccgggacccggaggtgctgcgtttgctccgc

SEQ ID NO: 12 - protein, 292 aa, Artificial sequence
  PLS3(F258V) inactive PLS3 mutant of PLS3-alpha.
MAGYLPPKGYAPSPPPPYPVTPGYPEPALHPGPGQAPVPAQVPAPAPGFALFPSPGPVALGSAAPFLPLPGVPSGLEFLVQIDQILIHQKAERVETFLGWETCNRYELRSGAGQPLGQAAEESNCCARLCCGARRPLRVRLADPGDREVLRLLRPLHCGCSCCPCGLQEMEVQAPPGTTIGHVLQTWHPFLPKFSIQDADRQTVLRVVGPCWTCGCGTDTNFEVKTRDESRSVGRISKQWGGLVREALTDADDFGLQVPLDLDVRVKAVLLGATFLIGPAFSWCLLRFLCLM

SEQ ID NO: 13 - protein, 295 aa, Artificial Sequence
  PLS3(F258V) inactive PLS3 mutant of PLS3-beta.
MAGYLPPKGYAPSPPPPYPVTPGYPEPALHPGPGQAPVPAQVPAPAPGFALFPSPGPVALGSAAPFLPLPGVPSGLEFLVQIDQILIHQKAERVETFLGWETCNRYELRSGAGQPLGQAAEESNCCARLCCGARRPLRVRLADPGDREVLRLLRPLHCGCSCCPCGLQEMEVQAPPGTTIGHVLQTWHPFLPKFSIQDADRQTVLRVVGPCWTCGCGTDTNFEVKTRDESRSVGRISKQWGGLVREALTDADDFGLQVPLDLDVRVKAVLLGATFLIDYMFFEKRGGAGPSAITS